SEQUENCE LISTING (135 sequences)

SEQ ID NO: 1 - protein, 6 aa, Artificial Sequence
  Synthentic peptides
AKLSIG

SEQ ID NO: 2 - protein, 5 aa, Artificial Sequence
  Synthetic peptide
QDXGX

SEQ ID NO: 3 - protein, 5 aa, Artificial Sequence
  Synthetic peptide
XQDXG

SEQ ID NO: 4 - protein, 6 aa, Artificial Sequence
  Synthetic peptide
XQDXGX

SEQ ID NO: 5 - protein, 13 aa, Artificial Sequence
  Synthetic peptide
XXXXQDXGXXXXX

SEQ ID NO: 6 - protein, 13 aa, Artificial Sequence
  Synthetic peptide
XCVXQDXGXHRCX

SEQ ID NO: 7 - protein, 13 aa, Artificial Sequence
  Synthetic peptide
XCVXQDXGXHRCX

SEQ ID NO: 8 - protein, 13 aa, Artificial Sequence
  Synthetic; compstatin
ICVVQDWGHHRCT

SEQ ID NO: 9 - protein, 13 aa, Artificial Sequence
  Synethetic; compstatin analog
ICVVQDWGHHRCT

SEQ ID NO: 10 - protein, 13 aa, Artificial Sequence
  Synthetic; compstatin analog
ICVYQDWGAHRCT

SEQ ID NO: 11 - protein, 13 aa, Artificial Sequence
  Synthetic; compstatin analog
ICVWQDWGAHRCT

SEQ ID NO: 12 - protein, 13 aa, Artificial Sequence
  Synthetic; compstatin analog
ICVWQDWGAHRCT

SEQ ID NO: 13 - protein, 13 aa, Artificial Sequence
  Synthetic; compstatin analog
ICVWQDWGAHRCT

SEQ ID NO: 14 - protein, 13 aa, Artificial Sequence
  Synthetic; compstatin analog
ICVAQDWGAHRCT

SEQ ID NO: 15 - protein, 13 aa, Artificial Sequence
  Synthetic; compstatin analog
ICVAQDWGAHRCT

SEQ ID NO: 16 - protein, 13 aa, Artificial Sequence
  Synthetic; compstatin analog
ICVAQDWGAHRCT

SEQ ID NO: 17 - protein, 13 aa, Artificial Sequence
  Synthetic; compstatin analog
ICVGQDWGAHRCT

SEQ ID NO: 18 - protein, 13 aa, Artificial Sequence
  Synthetic; compstatin analog
ICVGQDWGAHRCT

SEQ ID NO: 19 - protein, 13 aa, Artificial Sequence
  Synthetic; compstatin analog
ICVWQDWGAHRCT

SEQ ID NO: 20 - protein, 13 aa, Artificial Sequence
  Synthetic; compstatin analog
ICVAQDWGAHRCT

SEQ ID NO: 21 - protein, 13 aa, Artificial Sequence
  Synthetic; compstatin analog
ICVAQDWGAHRCT

SEQ ID NO: 22 - protein, 13 aa, Artificial Sequence
  Synthetic; compstatin analog
ICVAQDWGAHRCT

SEQ ID NO: 23 - protein, 13 aa, Artificial Sequence
  Synthetic; compstatin analog
ICVAQDWGAHRCT

SEQ ID NO: 24 - protein, 13 aa, Artificial Sequence
  Synthetic; compstatin analog
ICVWQDWGXHRCT

SEQ ID NO: 25 - protein, 16 aa, Artificial Sequence
  Synthetic; compstatin analog
GICVWQDWGAHRCTAN

SEQ ID NO: 26 - protein, 13 aa, Artificial Sequence
  Synthetic; compstatin analog
ICVWQDWGAHRCT

SEQ ID NO: 27 - protein, 13 aa, Artificial Sequence
  Synthetic; compstatin analog
ICVWQDWGAHRCT

SEQ ID NO: 28 - protein, 13 aa, Artificial Sequence
  Synthetic; compstatin analog
ICVWQDWGAHRCT

SEQ ID NO: 29 - protein, 13 aa, Artificial Sequence
  Synthetic; compstatin analog
ICVWQDWGAHRCT

SEQ ID NO: 30 - protein, 13 aa, Artificial Sequence
  Synthetic; compstatin analog
ICVWQDWGAHRCT

SEQ ID NO: 31 - protein, 13 aa, Artificial Sequence
  Synthetic; compstatin analog
ICVWQDWGAHRCT

SEQ ID NO: 32 - protein, 13 aa, Artificial Sequence
  Synthetic; compstatin analog
ICVWQDWGAHRCT

SEQ ID NO: 33 - protein, 10 aa, Artificial Sequence
  Synthetic peptide
ISHKDMQLGR

SEQ ID NO: 34 - protein, 10 aa, Artificial Sequence
  Synthetic peptide
YSFKDMQLGR

SEQ ID NO: 35 - protein, 10 aa, Artificial Sequence
  Synthetic peptide
YSFKDMPLXR

SEQ ID NO: 36 - protein, 10 aa, Artificial Sequence
  Synthetic peptide
YSFKPMPLXR

SEQ ID NO: 37 - protein, 21 aa, Artificial Sequence
  Synthetic peptide
RAARISLGPRXYSFKPMPLXR

SEQ ID NO: 38 - protein, 20 aa, Artificial Sequence
  Synthetic peptide
KYKHSVVKKXYSFKPMPLXR

SEQ ID NO: 39 - protein, 6 aa, Artificial Sequence
  Synthetic peptide
FKPXWR

SEQ ID NO: 40 - protein, 6 aa, Artificial Sequence
  Synthetic peptide
FKPXWR

SEQ ID NO: 41 - protein, 6 aa, Artificial Sequence
  Synthetic peptide
FKPXWR

SEQ ID NO: 42 - protein, 6 aa, Artificial Sequence
  Synthetic peptide
FKPLWR

SEQ ID NO: 43 - protein, 6 aa, Artificial Sequence
  Synthetic peptide
FKPXWR

SEQ ID NO: 44 - protein, 6 aa, Artificial Sequence
  Synthetic peptide
FXPXWR

SEQ ID NO: 45 - protein, 5 aa, Artificial Sequence
  Synthetic peptide
FPXWR

SEQ ID NO: 46 - protein, 5 aa, Artificial Sequence
  Synthetic peptide
FPXWR

SEQ ID NO: 47 - protein, 5 aa, Artificial Sequence
  Synthetic peptide
FPXWR

SEQ ID NO: 48 - protein, 5 aa, Artificial Sequence
  Synthetic peptide
FPXWR

SEQ ID NO: 49 - protein, 6 aa, Artificial Sequence
  Synthetic peptide
FKPXWR

SEQ ID NO: 50 - protein, 6 aa, Artificial Sequence
  Synthetic peptide
FXPXWR

SEQ ID NO: 51 - protein, 6 aa, Artificial Sequence
  Synthetic peptide
FWPXWR

SEQ ID NO: 52 - protein, 6 aa, Artificial Sequence
  Synthetic peptide
FKMXWR

SEQ ID NO: 53 - protein, 6 aa, Artificial Sequence
  Synthetic peptide
FKKXWR

SEQ ID NO: 54 - protein, 5 aa, Artificial Sequence
  Synthetic peptide
FPXWR

SEQ ID NO: 55 - protein, 5 aa, Artificial Sequence
  Synthetic peptide
FPXWR

SEQ ID NO: 56 - protein, 7 aa, Artificial Sequence
  Synthetic peptide
KFXPXWR

SEQ ID NO: 57 - protein, 6 aa, Artificial Sequence
  Synthetic peptide
FXPXWR

SEQ ID NO: 58 - protein, 6 aa, Artificial Sequence
  Synthetic peptide
FKPXWR

SEQ ID NO: 59 - protein, 6 aa, Artificial Sequence
  Synthetic peptide
FXPXWR

SEQ ID NO: 60 - protein, 6 aa, Artificial Sequence
  Synthetic peptide
FKPXWR

SEQ ID NO: 61 - protein, 6 aa, Artificial Sequence
  Synthetic peptide
FXPXWR

SEQ ID NO: 62 - protein, 6 aa, Artificial Sequence
  Synthetic peptide
FXPXWR

SEQ ID NO: 63 - protein, 6 aa, Artificial Sequence
  Synthetic peptide
FXPXWR

SEQ ID NO: 64 - protein, 6 aa, Artificial Sequence
  Synthetic peptide
FXPXWR

SEQ ID NO: 65 - protein, 6 aa, Artificial Sequence
  Synthetic peptide
FXPXWR

SEQ ID NO: 66 - protein, 7 aa, Artificial Sequence
  Synthetic peptide
XFXPXWR

SEQ ID NO: 67 - protein, 6 aa, Artificial Sequence
  Synthetic peptide
FXPXWR

SEQ ID NO: 68 - protein, 6 aa, Artificial Sequence
  Synthetic peptide
FXPXWR

SEQ ID NO: 69 - protein, 6 aa, Artificial Sequence
  Synthetic peptide
GXPXWR

SEQ ID NO: 70 - protein, 6 aa, Artificial Sequence
  Synthetic peptide
XXPXWR

SEQ ID NO: 71 - protein, 6 aa, Artificial Sequence
  Synthetic peptide
XXPXWR

SEQ ID NO: 72 - protein, 6 aa, Artificial Sequence
  Synthetic peptide
XXPXWR

SEQ ID NO: 73 - protein, 6 aa, Artificial Sequence
  Synthetic peptide
FXPXWR

SEQ ID NO: 74 - protein, 6 aa, Artificial Sequence
  Synthetic peptide
FXPXWR

SEQ ID NO: 75 - protein, 6 aa, Artificial Sequence
  Synthetic peptide
YXPXWR

SEQ ID NO: 76 - protein, 6 aa, Artificial Sequence
  Synthetic peptide
HXPXWR

SEQ ID NO: 77 - protein, 5 aa, Artificial Sequence
  Synthetic peptide
XPXWR

SEQ ID NO: 78 - protein, 5 aa, Artificial Sequence
  Synthetic peptide
XPXWR

SEQ ID NO: 79 - protein, 6 aa, Artificial Sequence
  Synthetic peptide
FXVXWR

SEQ ID NO: 80 - protein, 6 aa, Artificial Sequence
  Synthetic peptide
FXFXWR

SEQ ID NO: 81 - protein, 6 aa, Artificial Sequence
  Synthetic peptide
FXIXWR

SEQ ID NO: 82 - protein, 6 aa, Artificial Sequence
  Synthetic peptide
FXXXWR

SEQ ID NO: 83 - protein, 6 aa, Artificial Sequence
  Synthetic peptide
FXXXWR

SEQ ID NO: 84 - protein, 6 aa, Artificial Sequence
  Synthetic peptide
FXPXWR

SEQ ID NO: 85 - protein, 6 aa, Artificial Sequence
  Synthetic peptide
FXPGWR

SEQ ID NO: 86 - protein, 6 aa, Artificial Sequence
  Synthetic peptide
FXPXWR

SEQ ID NO: 87 - protein, 6 aa, Artificial Sequence
  Synthetic peptide
FXPXWR

SEQ ID NO: 88 - protein, 6 aa, Artificial Sequence
  Synthetic peptide
FXPXWR

SEQ ID NO: 89 - protein, 6 aa, Artificial Sequence
  Synthetic peptide
FXPXWR

SEQ ID NO: 90 - protein, 6 aa, Artificial Sequence
  Synthetic peptide
FXPXWR

SEQ ID NO: 91 - protein, 6 aa, Artificial Sequence
  Synthetic peptide
FXPXWR

SEQ ID NO: 92 - protein, 6 aa, Artificial Sequence
  Synthetic peptide
FXPXWR

SEQ ID NO: 93 - protein, 6 aa, Artificial Sequence
  Synthetic peptide
FXPXWR

SEQ ID NO: 94 - protein, 6 aa, Artificial Sequence
  Synthetic peptide
FXPXWR

SEQ ID NO: 95 - protein, 6 aa, Artificial Sequence
  Synthetic peptide
FXPXWR

SEQ ID NO: 96 - protein, 6 aa, Artificial Sequence
  Synthetic peptide
FXPXWR

SEQ ID NO: 97 - protein, 6 aa, Artificial Sequence
  Synthetic peptide
FXPXWR

SEQ ID NO: 98 - protein, 6 aa, Artificial Sequence
  Synthetic peptide
FXPXLR

SEQ ID NO: 99 - protein, 6 aa, Artificial Sequence
  Synthetic peptide
FXPXXR

SEQ ID NO: 100 - protein, 6 aa, Artificial Sequence
  Synthetic peptide
FXPXHR

SEQ ID NO: 101 - protein, 6 aa, Artificial Sequence
  Synthetic peptide
FXPXFR

SEQ ID NO: 102 - protein, 6 aa, Artificial Sequence
  Synthetic peptide
FXPXXR

SEQ ID NO: 103 - protein, 6 aa, Artificial Sequence
  Synthetic peptide
FXPXFR

SEQ ID NO: 104 - protein, 6 aa, Artificial Sequence
  Synthetic peptide
FXPXXR

SEQ ID NO: 105 - protein, 6 aa, Artificial Sequence
  Synthetic peptide
FXPXXR

SEQ ID NO: 106 - protein, 6 aa, Artificial Sequence
  Synthetic peptide
FXPXXR

SEQ ID NO: 107 - protein, 6 aa, Artificial Sequence
  Synthetic peptide
FXPXXR

SEQ ID NO: 108 - protein, 6 aa, Artificial Sequence
  Synthetic peptide
FXPXXR

SEQ ID NO: 109 - protein, 6 aa, Artificial Sequence
  Synthetic peptide
FXPXXR

SEQ ID NO: 110 - protein, 6 aa, Artificial Sequence
  Synthetic peptide
FXPXWX

SEQ ID NO: 111 - protein, 6 aa, Artificial Sequence
  Synthetic peptide
FXPXWX

SEQ ID NO: 112 - protein, 6 aa, Artificial Sequence
  Synthetic peptide
FXPXWK

SEQ ID NO: 113 - protein, 6 aa, Artificial Sequence
  Synthetic peptide
FXPXFR

SEQ ID NO: 114 - protein, 6 aa, Artificial Sequence
  Synthetic peptide
FXPXXR

SEQ ID NO: 115 - protein, 6 aa, Artificial Sequence
  Synthetic peptide
FXPXYR

SEQ ID NO: 116 - protein, 6 aa, Artificial Sequence
  Synthetic peptide
FXPXFX

SEQ ID NO: 117 - protein, 6 aa, Artificial Sequence
  Synthetic peptide
FXPXFX

SEQ ID NO: 118 - protein, 6 aa, Artificial Sequence
  Synthetic peptide
FXPXFX

SEQ ID NO: 119 - protein, 6 aa, Artificial Sequence
  Synthetic peptide
FXPXFR

SEQ ID NO: 120 - protein, 6 aa, Artificial Sequence
  Synthetic peptide
FXPGXR

SEQ ID NO: 121 - protein, 6 aa, Artificial Sequence
  Synthetic peptide
FXPXWR

SEQ ID NO: 122 - protein, 6 aa, Artificial Sequence
  Synthetic peptide
FXPXAR

SEQ ID NO: 123 - protein, 6 aa, Artificial Sequence
  Synthetic peptide
FXPAWR

SEQ ID NO: 124 - protein, 6 aa, Artificial Sequence
  Synthetic peptide
FXAXWR

SEQ ID NO: 125 - protein, 6 aa, Artificial Sequence
  Synthetic peptide
AXPXWR

SEQ ID NO: 126 - protein, 6 aa, Artificial Sequence
  Synthetic peptide
AXPXWR

SEQ ID NO: 127 - protein, 6 aa, Artificial Sequence
  Synthetic peptide
FXPXWR

SEQ ID NO: 128 - protein, 5 aa, Artificial Sequence
  Synthetic peptide
XPXWR

SEQ ID NO: 129 - protein, 5 aa, Artificial Sequence
  Synthetic peptide
XPXWR

SEQ ID NO: 130 - protein, 10 aa, Artificial Sequence
  Synthetic peptide
WESNGQPENN

SEQ ID NO: 131 - protein, 17 aa, Artificial Sequence
  Synthetic peptide
KTISKAKGQPREPQVYT

SEQ ID NO: 132 - protein, 15 aa, Artificial Sequence
  Synthetic; compstatin analog
GICVWQDWGAHRCTN

SEQ ID NO: 133 - protein, 13 aa, Artificial Sequence
  Synthetic; compstatin analog
ICVWQDWGAHRCT

SEQ ID NO: 134 - protein, 13 aa, Artificial Sequence
  Synthetic; compstatin analog
ICVWQDWGAHRCT

SEQ ID NO: 135 - protein, 15 aa, Artificial Sequence
  Synthetic; compstatin analog
GICVWQDWGAHRCTN

(19) United States
(12) Patent Application Publication
(10) Pub. No.: US 2011/0092446 A1
(43) Pub. Date: Apr. 21, 2011

(54) COMPOSITIONS AND METHODS FOR TREATMENT OF TRAUMA

(76) Inventors: Cedric Francois, Louisville, KY (US); Pascal Deschatelets, Louisville, KY (US); Paul Olson, Louisville, KY (US)

(21) Appl. No.: 12/669,957

(22) PCT Filed: Jul. 21, 2008
(86) PCT No.: PCT/US2008/070650

§ 371 Date: Oct. 18, 2010

Related U.S. Application Data
(60) Provisional application No. 60/951,048, filed on Jul. 20, 2007.
(60) Provisional application No. 60/992,737, filed on Dec. 6, 2007.

(51) Int. Cl. A61K 38/12; C12Q 1/68; C07K 7/64

(52) U.S. Cl. 514/21.1; 435/6; 530/321

(57) ABSTRACT

The present invention features the use of a complement inhibitor, e.g., a compstatin analog for treating an individual who has suffered a severe injury. In some embodiments, the complement inhibitor may be administered within 24 hours following the injury and optionally also at later time points. The complement inhibitor may, for example, be administered prior to transporting the patient to a health care facility, during transport of the patient to a health care facility, or in the emergency department. Further provided are methods of selecting individuals for such therapy. Further provided are methods of identifying individuals at increased risk of poor outcome following trauma. In certain embodiments the methods comprise determining whether the genotype of the patient includes an allele of a polymorphism in or near a complement-related gene, wherein said allele is associated with risk of poor outcome following trauma.

### CROSS-REFERENCE TO RELATED APPLICATIONS

This application claims priority to and the benefit of U.S. provisional patent applications U.S. Ser. No. 60/951,048, filed Jul. 20, 2007, and U.S. Ser. No. 60/992,737, filed Dec. 6, 2007, both of which are incorporated herein by reference.

### BACKGROUND OF THE INVENTION

Trauma patient usually refers to someone who has suffered serious physical injury such as open wounds, blunt injury, and major burns, potentially resulting in secondary complications such as shock, respiratory failure, sepsis, long-term or permanent disability, and death. Trauma patients often require specialized care, such as surgery and sometimes blood transfusion, within the so-called golden hour of emergency medicine, the first sixty minutes after trauma occurs. This is not a strict deadline, but recognizes that many deaths which could have been prevented by appropriate care occur a relatively short time after injury.

Traumatic injury is a worldwide problem affecting individuals of all ages and socioeconomic backgrounds. Its causes are equally diverse, ranging from motor vehicle crashes (41% of cases) to burns and falls (27% of cases), firearm-related events (6% of cases) and other interpersonal violence (6% of cases) to motor vehicle crashes (41% of cases), burns and falls (27% of cases) (U.S. data, National Trauma Data Bank Report 2006, version 6.0) An estimated 5 million people died as a result of injury in 2000, representing approximately 9% of worldwide deaths and an estimated 12-16% of the global disease burden. In the U.S. trauma is the fourth leading cause of death overall, accounting for over 160,000 deaths, and the leading cause of death among those aged 1-44 years. At least 1.4 million Americans experience a traumatic brain injury every year, resulting in 235,000 hospitalizations, 50,000 deaths, and 80,000 to 90,000 individuals suffering from permanent impairment. Burns result in approximately 100,000 hospitalizations and 5,000 deaths annually in the U.S. alone.

The social and economic burdens of traumatic injury are immense. In 2004, over $117 billion, or 10% of total medical expenditures, was spent on injury-related medical care in the U.S. (Centers for Disease Control and Prevention (CDC). MMWR Morb Mortal Wkly Rep., 53(1):1-4, 2004). The actual economic cost of trauma is much larger, in part because it affects individuals in a relatively younger segment of the population as compared with other leading causes of death and disability, resulting in significant lost productivity and work years as well as long-term care needs.

The clinical conditions associated with severe trauma include hemorrhagic shock and resuscitation, ischemia-reperfusion injury, acute respiratory distress syndrome (ARDS), thermal injury, inhalation injury, sepsis, and traumatic brain injury. Mortality rates for the injured vary significantly depending on injury severity, presence of shock or central nervous system injury, physiologic reserve, and availability of appropriate care Immediate and early (within 24 hours) trauma deaths are due largely to primary central nervous system injury (˜40-50% of fatalities) and significant blood loss (˜30-40% of fatalities). During subsequent hospitalization, death from causes not directly related to specific injuries becomes more common. Systemic inflammatory response syndrome (SIRS), multi-organ failure (MOF), secondary brain damage, and infection are the major causes of death in the hospitalized trauma patient after the first 24-48 hours. Central nervous system (CNS) and respiratory system dysfunction are major contributors to early lethality after trauma. Secondary brain injuries and impairment of the hepatic, renal, gastrointestinal, hematopoietic, and cardiovascular systems typically manifest somewhat later. Post-traumatic ARDS and respiratory failure can occur early after injury in association with hemorrhagic shock or later in association with multiple organ system injury and pneumonia. Mortality due to MOF increases dramatically with the number of organ systems affected. With appropriate therapy, mortality is usually under 5% with single organ failure, increasing to 90% or more when at least four organ systems fail.

In the developed world, civilian trauma care provision is integrated from the level of field emergency service providers through outlying hospitals to specialized trauma care centers to which severely injured patients are transported following initial stabilization. Initial prehospital care focuses on maintaining adequate airway patency, breathing, and circulation. Acute management of traumatic hemorrhage can be divided into the resuscitative, operative, and critical care phases, geared towards control of bleeding, prevention of coagulopathy, and hemodynamic stabilization. Injury-specific measures include wound care, mechanical ventilation in the case of severe chest trauma or pulmonary dysfunction, surgery for fractures, repair of vascular injuries, and removal of penetrating objects. Management of severe traumatic central nervous system injury according to established guidelines has been shown to improve survival and functional outcomes. In the hospital setting, attention is paid to monitoring and correcting raised intracranial pressure.

Thus much post-trauma care is surgical and supportive in nature. Prophylactic antibiotics have a role in some contexts, particularly in abdominal trauma and burns. Recombinant activated coagulation factor VII (rFVIIa) is increasingly being administered to massively bleeding trauma patients. Some studies suggest that it benefits some patient populations as reflected in reduced need for transfusion and reduced mortality and critical complications. Pharmacological therapies for traumatic brain injury and acute spinal cord injury are limited. There is a need in the art for new approaches to treating trauma patients.

### SUMMARY OF THE INVENTION

The invention provides a method of treating an individual who has suffered a traumatic injury comprising administering a complement inhibitor to the individual. In certain embodiments of the invention the complement inhibitor is administered within 24 hours following the injury, e.g., within 30 minutes, within 1, 2, 3, 4, 6,or 8 hours following the injury. In some embodiments the complement inhibitor is a compstatin analog. In some embodiments the individual has a genotype associated with increased susceptibility to a complement-mediated disorder relative to members of the general population (e.g., relative to individuals that are reasonably matched with respect to one or more demographic factors such as age, sex, etc.) but that have a different genotype. In some embodiments the individual has a genotype associated with decreased susceptibility to a complement-mediated disorder (e.g., relative to members of the general population that are reasonably matched with respect to one or more demographicfactors such as age, sex, etc.) but that have a different genotype.

In some embodiments of the invention, the genotype of the individual comprises a haplotype associated with increased susceptibility to a complement-mediated disorder, wherein said haplotype comprises at least one allele of a gene encoding a complement-related protein. In some embodiments of the invention, the genotype of the individual comprises a haplotype associated with decreased susceptibility to a complement-mediated disorder, wherein said haplotype comprises at least one allele of a gene encoding a complement-related protein. In some embodiments the genotype of the individual comprises an allele of a gene encoding a complement-related protein, wherein the allele is associated with increased susceptibility to a complement-mediated disorder relative to at least some other alleles of said gene. In some embodiments the genotype of the individual comprises an allele of a gene encoding a complement-related protein, wherein the allele is associated with decreased susceptibility to a complement-mediated disorder relative to at least some other alleles of said gene. In some embodiments the individual is heterozygous for the allele. In some embodiments the individual is homozygous for the allele.

In some embodiments the genotype comprises a nucleotide at a polymorphic site, wherein presence of the nucleotide at the polymorphic site is associated with increased susceptibility to a complement-mediated disorder relative to the susceptibility that would exist if an alternate nucleotide was present at the polymorphic site. In some embodiments of the invention the genotype comprises a nucleotide at a polymorphic site, wherein presence of the nucleotide at the polymorphic site is associated with decreased susceptibility to a complement-mediated disorder relative to the susceptibility that would exist if an alternate nucleotide was present at the polymorphic site.

A polymorphic site or polymorphism of interest herein may be in a gene encoding a complement-related protein or may be located near such a gene (e.g., in an intergenic region). The polymorphism may or may not alter the sequence of an encoded protein. Polymorphisms may, for example, alter gene expression (transcription, splicing, nuclear export, stability, translation, etc.). Polymorphisms that alter the sequence of a protein may additionally or alternately alter one or more properties of the encoded protein such as enzymatic activity, localization, stability, physical interactions, etc.

In some embodiments the complement-related protein is a complement component. In some embodiments the complement-related protein is a receptor for a soluble complement component. In some embodiments the complement-related protein is a complement regulatory protein. In some embodiments the complement-related protein is a complement control protein. In some embodiments the complement-related protein is a complement-like protein. In some embodiments the complement-related protein is selected from: complement factor H (CFH), complement component C2 (C2), factor B, complement component C7 (C7), complement component C3 (C3), and mannose binding lectin 2 (MBL-2). In some embodiments the complement-related protein is selected from C1, C2, C3, C4, C5, C6, C7, C8, C9, C5a receptor (C5aR), C3a receptor (C3aR), Complement Receptor 1 (CR1), Complement Receptor 2 (CR2), Complement Receptor 3 (CR3), factor B, D, I, properdin, MBL-1, MBL-2, Clinh, C4 binding protein, CD46, CD55, CD34, CD59, vitronectin, clusterin, MASP-1, MASP-2, CFHR1, CFHR2, CFHR3, CFHR4, and CFHR5. In some embodiments the gene does not encode a complement-related protein but instead encodes an endogenous protein that physically interacts with a complement-related protein. One example of such a protein is Protein C. In some embodiments the haplotype, allele and/or polymorphism is/are associated with increased susceptibility to a disease selected from the group consisting of age-related macular degeneration (AMD), atypical hemolytic uremic syndrome (aHUS), paroxysmal nocturnal hemoglobinuria, membranoproliferative nephritis, and cardiovascular disease. In some embodiments the haplotype, allele and/or polymorphism is/are associated with decreased susceptibility to a disease selected from the group consisting of age-related macular degeneration (AMD), atypical hemolytic uremic syndrome (aHUS)paroxysmal nocturnal hemoglobinuria, membranoproliferative nephritis, and cardiovascular disease.

In some embodiments the polymorphism alters the coding sequence of the complement-related protein. In some embodiments the polymorphism results in altered expression level of the complement-related protein. In some embodiments the polymorphism results in altered activity or localization of the complement-related protein. In some embodiments the polymorphism alters the electrophoretic mobility of the complement-related protein. In some embodiments the polymorphism is a single nucleotide polymorphism (SNP). In some embodiments the polymorphism is selected from: polymorphisms (Tyr402His, rs1061170), rs2274700, rs1061147, and rs7535263 in the CFH gene. In other embodiments the polymorphism is selected from any polymorphism described in Li, M, et al., Nat Genet, 38:1049-54, 2006 (see, Tables 1 and 2); Gold, B., et al., Nat. Genet, 38: 458-62, 2006; Dinu, V., et al., Genetic Epidemiology, 31: 224-237, 2007 (see, e.g., Tables 3 and 5), Yates, J. R. W., N. Engl. J. Med., 357: 19-27, 2007 (see, e.g., Tables 2 and 3), Francis, P., et al., PLoS ONE. November 28; 2(11):e1197, 2007 (see, e.g., Tables 1 and 2 therein), and/or other references mentioned herein, wherein the polymorphism is associated with an increased or decreased susceptibility to AMD. In some embodiments the polymorphism is a polymorphism in strong linkage disequilibrium with any of the afore-mentioned polymorphisms.

The invention provides a method of identifying a trauma patient as being at increased risk of poor outcome following trauma, the method comprising: (a) determining the genotype of the trauma patient with respect to one or more complement-related genes, wherein at least two alleles of the gene exist in the population, and wherein at least one allele is a risk allele for a poor outcome following trauma (“trauma risk allele”); and (b) identifying the patient as being at increased risk of poor outcome following trauma if the trauma patient's genotype comprises the trauma risk allele. In some embodiments of the invention the trauma patient is identified as being at increased risk of poor outcome if the patient is homozygous for the trauma risk allele. In some embodiments of the invention the trauma patient is identified as being at increased risk of poor outcome if the patient is heterozygous for the trauma risk allele. The method may further comprise making a clinical decision based at least in part on whether the patient is identified as being at increased risk of poor outcome. The clinical decision could be any decision or course of action taken by a health care provider that relates to or modifies the care provided to the patient. In some embodiments the clinical decision comprises administering a complement inhibitor. In some embodiments the clinical decision comprises administering an antibiotic. In some embodiments the clinical decision comprises ordering a laboratory test. The laboratory test could be, e.g., a test that provides information concerning the status of the complement system in the patient. For example, the test may determine the level of complement activation, the level of one or more complement-related proteins, etc. The test may assess the status of a body system, e.g., the cardiovascular system, respiratory system, etc. In some embodiments, a patient identified as being at increased risk is monitored more closely than would otherwise be the case.

The invention provides a method of selecting a trauma patient as a suitable candidate for therapy with a complement inhibitor, the method comprising: (a) determining the genotype of the trauma patient with respect to one or more complement-related genes, wherein at least two alleles of the gene exist in the population, and wherein at least one allele is a risk allele for a poor outcome following trauma; and (b) selecting the trauma patient as a suitable candidate for therapy with a complement inhibitor if the trauma patient is homozygous or heterozygous for the trauma risk allele. The method can further comprise administering a complement inhibitor to the trauma patient.

The invention further provides a method of selecting a therapeutic agent for a trauma patient, the method comprising: (a) determining the genotype of the trauma patient with respect to one or more genes, wherein at least two alleles of the gene exist in the population, and wherein at least one allele is a risk allele for developing AMD (“AMD risk allele”); and (b) selecting a complement inhibitor as a therapeutic agent for the trauma patient if the trauma patient is homozygous or heterozygous for the AMD risk allele. In some embodiments, a complement inhibitor is selected as a therapeutic agent for the trauma patient if the patient is heterozygous for the AMD risk allele but not if the patient is homozygous for the AMD risk allele. The invention further provides a method of selecting a therapeutic agent for a trauma patient, the method comprising: (a) determining the genotype of the trauma patient with respect to one or more genes, wherein at least two alleles of the gene exist in the population, and wherein at least one allele is an AMD risk allele; and (b) selecting a complement inhibitor as a therapeutic agent for the trauma patient if the trauma patient is not homozygous or heterozygous for the AMD risk allele.

The invention further provides a method of selecting a trauma patient as a suitable candidate for therapy with a complement inhibitor, the method comprising: (a) determining the genotype of the trauma patient with respect to one or more complement-related genes, wherein at least two alleles of the gene exist in the population, and wherein at least one allele is a risk allele for developing AMD; and (b) selecting the trauma patient as a suitable candidate for therapy with a complement inhibitor based at least in part on the genotype.

The invention further provides a method of treating a trauma patient comprising: (a) determining the genotype of the trauma patient with respect to one or more complement-related genes, wherein at least two alleles of the gene exist in the population, and wherein at least one allele is a risk allele for a poor outcome following trauma; and (b) administering a complement inhibitor to the trauma patient if the trauma patient is homozygous or heterozygous for the trauma risk allele. In some embodiments of the invention the trauma patient is homozygous for the trauma risk allele. In some embodiments of the invention the trauma patient is heterozygous for the trauma risk allele. In some embodiments of the invention the trauma risk allele is one wherein the odds ratio (OR) with respect to having a poor outcome following trauma in individuals heterozygous for the risk allele (compared with individuals not having a copy of the risk allele) is at least 1.2, while in some embodiments said OR is at least 1.5, and in some embodiments said OR is at least 2.0. In some embodiments of the invention the risk allele is one wherein the odds ratio (OR) with respect to having a poor outcome following trauma in individuals homozygous for the risk allele (compared with individuals not having a copy of the risk allele) is at least 1.2, while in some embodiments said OR is at least 1.5, and in some embodiments said OR is at least 2.0. In some embodiments the poor outcome is death. In some embodiments, the poor outcome is development of MOF.

The invention further provides a method of selecting a trauma patient as a suitable candidate for therapy with a complement inhibitor, the method comprising: (a) determining the genotype of the trauma patient with respect to one or more SNPs, wherein at least one of the SNPs is in or linked to a complement-related gene; and (b) selecting the trauma patient as a suitable candidate for therapy with a complement inhibitor based at least in part on the genotype. In some embodiments, at least one of the SNPs in or linked to a complement-related gene is associated with a complement-mediated disorder. In some embodiments, the patient is homozygous for an allele that is associated with increased risk of developing the disorder. In some embodiments, the patient is homozygous for an allele that is associated with decreased risk of developing the disorder. In some embodiments, the patient is heterozygous for an allele that is associated with increased risk of developing the disorder. In some embodiments, the patient is heterozygous for an allele that is associated with decreased risk of developing the disorder.

In some embodiments the method of treatment comprises selecting the particular complement inhibitor to be used based at least in part on the genotype of the individual. In some embodiments the method comprises selecting the dose of the complement inhibitor to be used based at least in part on the genotype of the individual.

In some embodiments of the methods of the invention that involve determining genotype with respect a complement-related gene, the genotype is determined with respect to a single polymorphism. In some embodiments of the methods of the invention that involve determining genotype with respect a complement-related gene, the genotype is determined with respect to multiple polymorphisms in the gene.

The methods are also of use, in certain embodiments of the invention, to select patients who would be more likely to benefit from complement inhibitor therapy in the context of surgeries such as cardiopulmonary bypass surgery, coronary artery bypass graft surgery (CABG), organ transplant, aneurysm repair, plastic surgery, flap surgery, and any surgical procedure in which tissue ischemia may occur, relative to at least some other patients. Thus the invention provides each of the above methods (and the other methods herein useful in the case of trauma patients), wherein the patient is a surgery patient rather than a trauma patient.

Further provided are primers, probes, related reagents, kits, devices, and systems useful for genotyping an individual with respect to polymorphisms that are associated with susceptibility to poor outcome following trauma. Further provided are methods of using the information gained from the genotype of a trauma patient with respect to one or more polymorphisms in or near a complement-related gene. One such method comprises using the information, optionally taking other information into account as well, in order to decide whether the patient is a suitable candidate for therapy with a complement inhibitor and/or selecting a complement inhibitor or dose based at least in part on the information. In some embodiments, individuals are rapidly genotyped (e.g., within 24 hours following trauma, or even within 1 hour following trauma). It is also envisioned that individuals will be genotyped as a routine matter, and the results stored in a database. The results would be provided, e.g., electronically, e.g., wirelessly, to EMS personnel or others involved in treating the trauma patient in the field. Alternately, the information could be carried by the individual in any of a variety of formats. Any means by which information regarding the genotype is obtained by care providers is within the scope of the invention.

As described herein, the invention provides a method of treating an individual who has suffered a traumatic injury comprising administering a complement inhibitor to the individual. In some embodiments the individual has a genotype associated with increased risk of poor outcome following trauma, and the complement inhibitor is administered so as to reduce the individual's risk of having a poor outcome. In some embodiments the individual's genotype comprises a trauma risk allele of a polymorphism, wherein the polymorphism is located in or near a gene that encodes a complement-related protein. In some embodiments the individual is one whose genotype with respect to one or more polymorphisms located in or near a gene that encodes a complement-related protein has been determined prior to administering the complement inhibitor. In some embodiments at least one of the polymorphisms is associated with risk of poor outcome following trauma. In some embodiments the complement inhibitor is administered within 24 hours following the injury. In some embodiments the complement inhibitor is administered within 1 hour following the injury. In some embodiments the complement inhibitor is administered in the field. In some embodiments the complement inhibitor inhibits both the classical and alternative pathways. In some embodiments the complement inhibitor inhibits only the alternative pathway. In some embodiments the complement inhibitor is administered in a fixed dose format. In some embodiments the complement inhibitor is administered intravenously. In some embodiments the method comprises administering the complement inhibitor as an IV bolus followed by an IV infusion. In some embodiments the method comprises administering the complement inhibitor as an IV bolus. In some embodiments the method comprises administering the complement inhibitor as an IV infusion. In some embodiments the method comprises administering the complement inhibitor as an IV drip. In some embodiments an IV bolus is administered over a time period of 10 seconds or less, e.g., 5 seconds or less. In some embodiments an IV infusion is administered over a time period of more than 10 seconds e.g., 15, 20, 30, or 60 seconds. In some embodiments an IV infusion is administered over a time period of more than 60 seconds e.g., 2, 3, 4, 5, 15, 20, 30, or 60 minutes. In some embodiments an IV drip is administered over more than 60 minutes, e.g., over about 2, 3, 5, 10, 15, 20, or more hours. In some embodiments the complement inhibitor is administered directly into the spinal canal or intracranial space. In some embodiments the individual has an injury severity score of at least 15. In some embodiments the individual has an injury severity score of at least 9. In some embodiments the individual has a revised trauma score of at least 4. In some embodiments the complement inhibitor is a compstatin analog. In some embodiments, the method further comprises: (i) determining the genotype of the individual with respect to a complement-related gene, wherein at least two alleles of the gene exist in the population, and wherein at least one allele is a risk allele for a poor outcome following trauma; and; and (ii) determining, based at least in part on said genotype, that the individual is a suitable candidate for administration of a complement inhibitor. In some embodiments the individual is determined to be a suitable candidate for administration of a complement inhibitor if the individual's genotype comprises an allele that is associated with increased susceptibility to AMD. In some embodiments the individual is determined to be a suitable candidate for administration of a complement inhibitor if the individual's genotype comprises an allele that is associated with decreased susceptibility to AMD. In some embodiments the genotype is determined with respect to a polymorphism selected from the group consisting of: rs1061170, rs1047286, rs2230199, rs120862610, rs9332739, rs547154, rs4151667, rs641153, rs41015361, rs33682798, rs10490924, and rs1045216. In some embodiments the gene encodes a protein selected from the group consisting of: complement factor H (CFH), complement proteins C2, C3, factor B, C7 and MBL-2.

The invention provides a method of identifying a gene associated with outcome following trauma, the method comprising: (a) determining the genotype of a plurality of individuals that had a poor outcome following a traumatic injury, wherein said genotype is determined with respect to a complement-related gene, wherein at least two alleles of the gene exist in the population; (b) determining the genotype of a plurality of control individuals that suffered a traumatic injury of similar severity and did not have a poor outcome, wherein said genotype is determined with respect to the gene or polymorphism of step (a); and (c) determining correlations between the genotype of the individuals and occurrence of a poor outcome, wherein one or more significant correlation(s) identifies the gene as being associated with outcome following trauma. In some embodiments the gene is a risk modifier for AMD. In some embodiments the gene encodes a complement protein. In some embodiments the gene encodes a complement control protein. In some embodiments a significant correlation between an allele of a gene and a poor outcome following trauma identifies the allele as being associated with a poor outcome following trauma.

The invention provides a method identifying an allele associated with outcome following trauma, the method comprising: (a) determining the genotype of a plurality of individuals that had a poor outcome following a traumatic injury, wherein said genotype is determined with respect to a complement-related gene or polymorphism in or near a complement-related gene, wherein at least two alleles of the gene exist in the population; (b) determining the genotype of a plurality of control individuals that did not have a poor outcome, wherein said genotype is determined with respect to the complement-related gene or polymorphism; and (c) determining whether one of the alleles of said gene or polymorphism is present at higher or lower frequency in individuals that had a poor outcome following traumatic injury relative to its frequency in controls, wherein: (i) presence of said allele at higher frequency in the individuals that had a poor outcome identifies the allele as being associated with poor outcome following trauma; (ii) presence of said allele at lower frequency in the individuals that had a poor outcome identifies said allele as being associated with a favorable outcome after traumatic injury; (iii) presence of said allele at higher frequency in the control individuals identifies the allele as being associated with favorable outcome following trauma; and/or (iv) presence of said allele at lower frequency in the control individuals identifies the allele as being associated with poor outcome following trauma. In some embodiments the gene is a risk modifier for AMD. In some embodiments the gene encodes a complement protein. In some embodiments the gene encodes a complement control protein. In some embodiments the controls are individuals who experienced a traumatic injury of similar nature and/or severity to that experienced by the individuals who had a poor outcome. In some embodiments the gene encodes CFH. In some embodiments the method comprises (a) determining the genotype of a plurality of individuals that had a poor outcome following a traumatic injury, wherein said genotype is determined with respect to a plurality of complement-related genes or polymorphisms in or linked to complement-related genes, wherein at least two alleles of the genes exist in the population; (b) determining the genotype of a plurality of control individuals that did not have a poor outcome, wherein said genotype is determined with respect to the complement-related genes or polymorphisms of (a); and (c) determining whether a combination of alleles of said genes or polymorphisms is present at higher or lower frequency in individuals that had a poor outcome following traumatic injury relative to the frequency at which said combination is present in controls, wherein: (i) presence of said combination of alleles at higher frequency in the individuals that had a poor outcome identifies the combination as being associated with poor outcome following trauma; (ii) presence of said combination of alleles at lower frequency in the individuals that had a poor outcome identifies said combination of alleles as being associated with a favorable outcome after traumatic injury; (iii) presence of said combination of alleles at higher frequency in the control individuals identifies the combination of alleles as being associated with favorable outcome following trauma; and/or (iv) presence of said combination of alleles at lower frequency in the control individuals identifies the combination of alleles as being associated with poor outcome following trauma. In some embodiments the genotype is determined with respect to at least two different complement-related genes or polymorphisms in or near at least two complement related genes. In some embodiments at least two of said polymorphisms are located in or near the same complement-related gene. In some embodiments one of said genes encodes CFH. In some embodiments one of said genes encodes C3.

The invention provides a method of identifying a polymorphic variant associated with outcome following trauma, the method comprising: (a) determining the genotype of a plurality of individuals that had a poor outcome following a traumatic injury, wherein said genotype is determined with respect to a polymorphic site in or linked to a complement-related gene; (b) determining the genotype of a plurality of control individuals that did not have a poor outcome, wherein said genotype is determined with respect to the same polymorphic site; and (c) determining whether one of the polymorphic variants of said gene is present at higher or lower frequency in individuals that had a poor outcome following traumatic injury relative to its frequency in controls, wherein: (i) presence of a polymorphic variant at higher frequency in the individuals that had a poor outcome identifies the polymorphic variant as being associated with poor outcome following trauma; (ii) presence of a polymorphic variant at lower frequency in the individuals that had a poor outcome identifies said polymorphic variant as being associated with a favorable outcome after traumatic injury; (iii) presence of a polymorphic variant at higher frequency in the control individuals identifies the polymorphic variant as being associated with favorable outcome following trauma; and/or (iv) presence of a polymorphic variant at lower frequency in the control individuals identifies the polymorphic variant as being associated with poor outcome following trauma. In some embodiments the gene is a risk modifier for AMD. In some embodiments the gene encodes a complement protein. In some embodiments the gene encodes a complement control protein. In some embodiments the polymorphic site is located in the gene. In some embodiments the controls are individuals who experienced a traumatic injury of similar nature and/or severity to that experienced by the individuals who had a poor outcome. In some embodiments the method comprises (a) determining the genotype of a plurality of individuals that had a poor outcome following a traumatic injury, wherein said genotype is determined with respect to a plurality of polymorphic sites in or near one or more complement-related gene(s); (b) determining the genotype of a plurality of control individuals that did not have a poor outcome, wherein said genotype is determined with respect to the polymorphic sites of step (a); and (c) determining whether a combination of polymorphic variants of the polymorphic sites of step (a) and (b) is present at higher or lower frequency in individuals that had a poor outcome following traumatic injury relative to the frequency at which said combination is present in controls, wherein: (i) presence of a combination of polymorphic variants at higher frequency in the individuals that had a poor outcome identifies the combination of polymorphic variants as being associated with poor outcome following trauma; (ii) presence of a combination of polymorphic variants at lower frequency in the individuals that had a poor outcome identifies said combination of polymorphic variants as being associated with a favorable outcome after traumatic injury; (iii) presence of a combination of polymorphic variants at higher frequency in the control individuals identifies the combination of polymorphic variants as being associated with favorable outcome following trauma; and/or (iv) presence of a combination of polymorphic variants at lower frequency in the control individuals identifies the combination of polymorphic variants as being associated with poor outcome following trauma. In some embodiments at least two of said polymorphic sites are located in or near different complement-related genes. In some embodiments at least two of said polymorphic sites are located in or near the same complement-related gene. In some embodiments at least one of said polymorphic sites is located in or near the gene that encodes CFH. In some embodiments at least one of said polymorphic sites is located in or near the gene that encodes C3.

The invention provides a method of selecting a therapeutic agent for a trauma patient, the method comprising: (a) determining the genotype of the trauma patient with respect to one or more polymorphisms in or near a complement related gene, wherein at least one allele of the polymorphism is a risk allele for a poor outcome following trauma; and

(b) selecting a complement inhibitor as a therapeutic agent for the trauma patient if the trauma patient is homozygous or heterozygous for the risk allele. In some embodiments the gene is a risk modifier for AMD. In some embodiments the gene encodes a complement protein. In some embodiments the gene encodes a complement control protein. In some embodiments the method further comprises administering a complement inhibitor to the trauma patient. In some embodiments the method further comprises administering a compstatin analog to the trauma patient. In some embodiments the gene encodes CFH. In some embodiments the gene encodes C3.

The invention provides a method of selecting a trauma patient as a suitable candidate for therapy with a complement inhibitor, the method comprising: (a) determining the genotype of the trauma patient with respect to a polymorphism in or near a complement-related gene, wherein at least one allele of the polymorphism is a risk allele for a poor outcome following trauma; and (b) selecting the trauma patient as a suitable candidate for therapy with a complement inhibitor if the trauma patient is homozygous or heterozygous for the risk allele. In some embodiments the gene is a risk modifier for AMD. In some embodiments the gene encodes a complement protein. In some embodiments the gene encodes a complement control protein. In some embodiments the method further comprises administering a complement inhibitor to the trauma patient. In some embodiments the method further comprises administering a compstatin analog to the trauma patient. In some embodiments the gene encodes CFH. In some embodiments the gene encodes C3.

The invention provides a method of selecting a therapeutic agent for a trauma patient, the method comprising: (a) determining the genotype of the trauma patient with respect to one or more genes or loci, wherein at least two alleles of the gene or locus exist in the population, and wherein at least one allele is a risk allele for developing a complement-mediated disorder, which is optionally AMD; and (b) selecting a complement inhibitor as a therapeutic agent for the trauma patient based at least in part on the genotype. In some embodiments the trauma patient is selected as a suitable candidate if the trauma patient is homozygous or heterozygous for the risk allele. In some embodiments the trauma patient is selected as a suitable candidate if the trauma patient is homozygous for the risk allele. In some embodiments the trauma patient is selected as a suitable candidate if the trauma patient is not homozygous or heterozygous for the risk allele. In some embodiments the trauma patient is selected as a suitable candidate if the trauma patient is not homozygous for the risk allele. In some embodiments the gene encodes a complement protein. In some embodiments the gene encodes a complement control protein. In some embodiments the method further comprises administering a complement inhibitor to the trauma patient. In some embodiments the method further comprises administering a compstatin analog to the trauma patient. In some embodiments the gene encodes CFH. In some embodiments the gene encodes C3.

The invention provides a method of selecting a trauma patient as a suitable candidate for therapy with a complement inhibitor, the method comprising: (a) determining the genotype of the trauma patient with respect to one or more complement-related genes, wherein at least two alleles of the gene exist in the population, and wherein at least one allele is a risk allele for developing a complement-mediated disorder, which is optionally AMD; and (b) selecting the trauma patient as a suitable candidate for therapy with a complement inhibitor based at least in part on the genotype. In some embodiments the trauma patient is selected as a suitable candidate if the trauma patient is homozygous or heterozygous for the risk allele. In some embodiments the trauma patient is selected as a suitable candidate if the trauma patient is homozygous for the risk allele. In some embodiments the trauma patient is selected as a suitable candidate if the trauma patient is not homozygous or heterozygous for the risk allele. In some embodiments the trauma patient is selected as a suitable candidate if the trauma patient is not homozygous for the risk allele. In some embodiments the gene encodes a complement protein. In some embodiments the gene encodes a complement control protein. In some embodiments the method further comprises administering a complement inhibitor to the trauma patient. In some embodiments the method further comprises administering a compstatin analog to the trauma patient. In some embodiments the step (a) comprises determining the genotype with respect to two or more polymorphisms each of which is in or near a complement-related gene. In some embodiments the gene encodes CFH. In some embodiments the gene encodes C3.

The invention provides a method of treating a trauma patient comprising: (a) determining the genotype of the trauma patient with respect to a polymorphism in or near a complement-related gene, wherein at least one allele of the polymorphism is a risk allele for developing AMD; and (b) administering a complement inhibitor to the trauma patient if the trauma patient is homozygous or heterozygous for the risk allele. In some embodiments the the gene encodes a complement protein. In some embodiments the gene encodes a complement control protein. In some embodiments the complement inhibitor is or comprises a compstatin analog. In some embodiments step (a) comprises determining the genotype with respect to two or more polymorphisms each of which is in or near a complement-related gene. In some embodiments the the polymorphisms are in or located closest to different complement-related genes. In some embodiments the polymorphism is in or near the gene that encodes CFH. In some embodiments the polymorphism is in or near the gene that encodes C3.

The invention provides a method of treating a trauma patient comprising: (a) determining the genotype of the trauma patient with respect to a polymorphism in or near a complement-related gene, wherein at least one allele of the polymorphism is a risk allele for developing AMD; and (b) administering a complement inhibitor to the trauma patient if the trauma patient is not homozygous or heterozygous for the risk allele. In some embodiments the gene encodes a complement protein. In some embodiments the complement inhibitor is or comprises a compstatin analog. In some embodiments step (a) comprises determining the genotype with respect to two or more polymorphisms each of which is in or near a complement-related gene. In some embodiments the polymorphisms are in or located closest to different complement-related genes. In some embodiments the gene encodes CFH. In some embodiments the gene encodes C3.

The invention provides a method of selecting a trauma patient as a suitable candidate for therapy with a complement inhibitor, the method comprising: (a) determining the genotype of the trauma patient with respect to one or more polymorphisms, wherein at least one variant of such polymorphism is associated with increased risk for developing a complement-mediated disorder; and (b) selecting the trauma patient as a suitable candidate for therapy with a complement inhibitor based at least in part on the genotype. In some embodiments the trauma patient is selected as a suitable candidate if the trauma patient is homozygous or heterozygous for the polymorphic variant associated with the increased risk. In some embodiments the trauma patient is selected as a suitable candidate if the trauma patient is not homozygous or heterozygous for the polymorphic variant associated with the increased risk. In some embodiments the polymorphism is in or linked to a gene that encodes a complement protein. In some embodiments the polymorphism is in or linked to a gene that encodes a complement control protein. In some embodiments the method further comprises administering a complement inhibitor to the trauma patient. In some embodiments the method further comprises administering a compstatin analog to the trauma patient. In some embodiments the complement-mediated disorder is AMD. In some embodiments the polymorphism is in or near the gene that encodes CFH. In some embodiments the polymorphism is in or near the gene that encodes C3. In some embodiments step (a) comprises determining the genotype with respect to two or more polymorphisms each of which is in or near a complement-related gene. In some embodiments the polymorphisms are in or located closest to different complement-related genes, e.g., CFH and C3. In some embodiments the method further comprises administering a complement inhibitor and a second agent effective to reduce the likelihood of poor outcome following trauma to the trauma patient.

The invention provides a method of identifying a trauma patient as being at increased risk of poor outcome following trauma, the method comprising: (a) determining the genotype of the trauma patient with respect to one or more polymorphisms each of which is in or near a complement-related gene, wherein at least one allele of the polymorphism is a risk allele for a poor outcome following trauma (“trauma risk allele”); and (b) identifying the patient as being at increased risk of poor outcome following trauma if the trauma patient's genotype comprises the trauma risk allele. In some embodiments the method further comprises making a clinical decision based at least in part on whether the patient is identified as being at increased risk of poor outcome. In some embodiments the gene encodes CFH. In some embodiments the gene encodes C3.

In some embodiments of any of the compositions or methods of the invention that involve at least one polymorphism, at least one polymorphism is a single nucleotide polymorphism (SNP) selected from: rs1061170, rs1047286, rs2230199, rs120862610, rs9332739, rs547154, rs4151667, rs641153, rs41015361, rs33682798, rs10490924, and rs1045216. The invention provides embodiments that relate to each of the afore-mentioned polymorphisms. Also provided are embodiments that relate to each combination of the afore-mentioned polymorphisms. In some embodiments of any of the compositions or methods of the invention that involve at least one allele (e.g., an allele of gene or a polymorphism such as a SNP), the allele of use in the composition or method is the major allele, wherein “major allele” refers to an allele that occurs more frequently within a population of individuals than the other alleles. In some embodiments of any of the compositions or methods of the invention that involve at least one allele (e.g., an allele of gene or a polymorphism such as a SNP), the allele of use in the composition or method is a minor allele, i.e., an allele that occurs less frequently than at least one other allele. In embodiments wherein more than two alleles exist, the minor allele of use in the composition or method may be the least common allele or may be an allele that is less common than the major allele but is more common than at least one other allele. In some embodiments of the invention alleles that are found with a frequency of less than 1% in a population are disregarded for purposes of determining the minor allele. The population of interest may be, e.g., individuals worldwide, individuals within a particular country, individuals within a particular group such as Europeans, Asians, Sub-Saharan Africans, or individuals descended from individuals in an afore-mentioned group.

In some embodiments of any of the compositions or methods of the invention involving a compstatin analog, the compstatin analog is selected from any compound or group of compounds (e.g., comprises any SEQ ID NO) described above in the section entitled “Compstatin Analogs”.

It will be understood that the invention provides the inventive methods and compositions wherein the individuals, e.g., trauma patients, are humans, and wherein the polymorphisms in or near complement-related genes are found in the human population. The invention also provides certain embodiments of the methods and compositions that relate to non-human animals, e.g., non-human primates, rodents (e.g., rabbits, mice). For example, it may be of use to test certain compositions and methods in animal models. In some embodiments, such non-human animals have a genotype associated with a poor outcome following trauma. The invention encompasses animal models containing a transgene encoding a variant of a complement-related gene, wherein the variant alters the sequence of the gene to a form that more closely resembles the sequence of a variant that, in humans, is associated with poor outcome following trauma and/or is associated with altered risk of a complement-mediated disorder. In some embodiments such animal models are developed using homologous recombination to replace an endogenous sequence with an altered form.

The invention further provides a complement inhibitor comprising a first cyclic peptide portion that inhibits C3 activation and a second cyclic peptide portion that comprises a C5a receptor antagonist. In some embodiments the first cyclic peptide portion comprises a compstatin analog and the second cyclic peptide portion comprises a sequence selected from SEQ ID NO: 57, 58, 77, and 78. In some embodiments the first cyclic peptide portion comprises a sequence selected from SEQ ID NO: 28, 29, 32, 34, 132, 133, and 135, and the second cyclic peptide portion comprises a sequence selected from SEQ ID NO: 57, 58, 77, and 78.

All articles, books, patent applications, patents, other publications, and electronic databases and resources (including sequences and information relating to SNPs) mentioned in this application are incorporated herein by reference. Art-accepted abbreviations for amino acids, genes, and other terms are used herein.

### Definitions

An “allele” is any of two or more alternative forms of a gene or more generally, any of two or more alternative forms of a portion of genomic DNA, which portion may be as little as a single nucleotide in length and may or may not be located within a gene. For example, the alternate forms of a polymorphism such as a single nucleotide polymorphism (SNP) are referred to in the art as “alleles”.

A “complement component” or “complement protein” is a protein that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, and properdin. Components of the lectin pathway include, e.g., MBL2, MASP-1, and MASP-2. Complement components also include cell-bound receptors for soluble complement components, wherein such receptor mediates one or more biological activities of such soluble complement component following binding of the soluble complement component. Such receptors include, e.g., C5a receptor (C5aR), C3a receptor (C3aR), Complement Receptor 1 (CR1), Complement Receptor 2 (CR2), Complement Receptor 3 (CR3, also known as CD45), etc. It will be appreciated that the term “complement component” is not intended to include those molecules and molecular structures that serve as “triggers” for complement activation, e.g., antigen-antibody complexes, foreign structures found on microbial or articifial surfaces, etc.

A “complement regulatory protein” is a protein involved in regulating complement activity. A complement regulatory protein may down-regulate complement activity by, e.g., inhibiting complement activation or by inactivating or accelerating decay of one or more activated complement proteins. Examples of complement regulatory proteins include C1 inhibitor, C4 binding protein, clusterin, vitronectin, CFH, factor I, and the cell-bound proteins CD46, CD55, CD59, CR1, CR2, and CR3.

A “complement control protein” is a complement regulatory protein comprising multiple SCR modules as described below. Examples include CFH, CD46, CD55, CR1, and CR2.

A “complement-like protein” is a protein that has significant sequence identity to a complement protein or a complement control protein over at least 20% of its length and/or specifically competes with the complement protein or complement control protein for binding to its target, e.g., has an affinity at least 10% as great. The genes encoding such proteins may be found in close proximity to genes encoding the complement protein or complement control protein having a similar sequence. For example, the CFH gene cluster contains numerous CFH-like genes (e.g., CFHR1, CFHR2, CFHR3, CFHR4, and CFHR5).

“Complement-related protein” refers collectively to complement components, complement regulatory proteins, and complement-like proteins; however, wherever the disclosure refers to complement-related proteins in general, it is understood that the invention encompasses embodiments that relate specifically to complement components, complement regulatory proteins, complement-like proteins, and any combination thereof. A “complement-related gene” is a gene that encodes a complement-related protein.

A “complement-mediated” disorder is a disorder, disease, or condition characterized in that complement activation plays a role in its development, progression, and/or symptom(s) and/or contributes to its severity. Examples include age-related macular degeneration (AMD), rheumatoid arthritis, systemic lupus erythematosus, hereditary angioedema, paroxysmal nocturnal hemoglobinurea, atypical hemolytic uremic syndrome, ischemia-reperfusion injury, Alzheimer's disease, asthma, and psoriasis.

The term “gene” is used as understood in the art. In mose cases a gene comprises a nucleic acid sequence that encodes a polypeptide and can also include intron sequences and other regions that are transcribed into RNA, regulatory sequences (e.g., promoters, enhancers), etc. It will be appreciated that a “gene” can encode a functional RNA such as a microRNA, tRNA, etc., rather than a polypeptide.

“Identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. By gap is meant a portion of a sequence that is not occupied by a residue. For example, the sequence A K L - - - SIG (SEQ ID NO: 1) contains a gap of three residues. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between a sequence of interest and sequences in any of a variety of public databases. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. See the Web site having URL www.ncbi.nlm.nih.gov for these programs. In a specific embodiment, percent identity of a sequence of interest and a second sequence is calculated using BLAST2 with default parameters.

“Plurality” means more than one.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. Certain non-limiting suitable analogs and modifications are described in WO2004026328. The polypeptide may be acetylated, e.g., at the N-terminus and/or amidated, e.g., at the C-terminus.

“Significant sequence identity” as applied to an amino acid sequence means that the sequence is at least approximately 20%, 30%, 40%, 50%, 60%, 70%, 80%, 90%, or 95% identical to a reference sequence. In specific embodiments the sequence is at least approximately 70%, 80%, 85%, 90%, 95%, 98%, or 99% identical to a reference sequence. In specific embodiments at least 20%, 30%, 40%, 50%, 60%, 70%, 80%, 90%, or 95% of the nonidentical amino acids are conservatively replaced relative to the reference sequence. Conservative replacements may be defined in accordance with Stryer, L., Biochemistry, 3rd ed., 1988, according to which amino acids in the following groups possess similar features with respect to side chain properties such as charge, hydrophobicity, aromaticity, etc. (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P, which may be considered to fall within group (1). In another accepted classification, conservative substitutions occur within the following groups: (1) Non-polar: A, L, I, V, G, P, F, W, M; (2) Polar: S, T, C, Y, N, Q. (3) Basic: K, R, H; (4) Acidic: D, E Amino acids with a small side chain (G, A, S, T, M) also form a group from among which conservative substitutions can be made. Other classification methods known in the art can be used. Furthermore, amino acid analogs and unnatural amino acids can be classified in accordance with these schemes.

“Trauma” or “traumatic injury” as used herein refers to a severe physical injury due to a cause other than surgery.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinaations of ranges and specific number of carbon atoms) therein, e.g., from about 1 to 7 carbon atoms. Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like. “Lower alkanoyl” refers to an optionally substituted straight or branched aliphatic acyclic residue having about 1 to about 5 carbon atoms (and all combinations and subcombinaations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and have from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O— group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### DETAILED DESCRIPTION OF CERTAIN EMBODIMENTS OF THE INVENTION

Overview

The present invention provides compositions and methods for treating an individual who has suffered a traumatic injury. Common causes of traumatic injury are motor vehicle accidents, falls, fires, interpersonal or self-inflicted violence (e.g., involving weapons such as guns or knives), occupational accidents, and combat-related events. In some embodiments, the methods comprise administering a complement inhibitor to the individual, e.g., within 24 hours following the injury. In almost all instances trauma occurs outside the setting of a health care facility. The complement inhibitor may be administered “in the field”, i.e., outside the setting of a health care facility, e.g., at or near the location where the injury occurred or during transport of the patient to a health care facility such as a hospital, clinic, or physician's office. Under such conditions it may be difficult to accurately assess the weight of the patient. The invention provides fixed dose formulations containing sufficient complement inhibitor to significantly inhibit complement activation in a child or adult human following a single administration. For example, such formulations could reduce systemic complement activity by between 50% and 99%, e.g., by at least 50%, 75%, or 90%, relative to levels present prior to administration or relative to normal, average levels.

As discussed below, complement is a system of cell-bound and serum proteins that constitute an important arm of the immune system. Under normal circumstances complement serves an important protective function. However, excessive complement activation can cause tissue damage. A number of studies have demonstrated that complement activation occurs after severe injury and appears to correlate with injury severity, with increased complement activation being predictive of worse outcomes. Several studies have suggested that complement activation is an important factor in many of the complications of severe trauma, contributing significantly to ischemia/reperfusion (FR) injury, adult respiratory distress syndrome (ARDS), multi-organ dysfunction syndrome (MODS), multi-organ failure (MOF), secondary central nervous system (CNS) injury, and sepsis. However, until the present invention these observations have not been translated into a useful therapeutic approach.

The present disclosure relates to polymorphisms associated with risk of poor outcome in trauma and the use of genotype information regarding such polymorphisms for providing diagnostic information relevant to trauma. The disclosure also relates to methods for selecting a trauma patient as a suitable candidate for therapy with a complement inhibitor (e.g., as being more likely to benefit from therapy with a complement inhibitor than at least some other trauma patients having different characteristics). The disclosure also relates to the use of genotype information regarding polymorphisms that are associated with risk of poor outcome following trauma for making a clinical decision, e.g., selecting a therapy, e.g., a complement inhibitor, for a trauma victim. The disclosure also relates to the use of genotype information regarding polymorphisms that are associated with risk of developing a complement-related disorder (e.g., a disorder other than trauma) for providing diagnostic information relevant to trauma, e.g., for identifying trauma patients as being at altered (e.g., increased or decreased) risk of poor outcome following trauma, relative to patients having a different genotype with respect to one or more such polymorphisms.

The present disclosure recognizes the utility of using polymorphisms previously identified as being associated with increased or decreased risk of a complement-mediated disorder for a variety of purposes in the context of trauma. Such purposes include (i) assessing the role of complement in trauma outcome; (ii) providing diagnostic information such as identifying trauma patients as being at increased or decreased risk of poor outcome following trauma (e.g., relative to members of the general population); (iii) predicting risk of poor outcome following trauma; (iv) selecting a trauma patient as a suitable candidate for therapy with a complement inhibitor; (v) selecting a therapy, e.g., a complement inhibitor, for a trauma patient.

Complement System

In order to facilitate an understanding of the invention, and without intending to limit the invention in any way, this section provides an overview of complement and its pathways of activation. Further details are found in the following references: Makrides, S C, Pharm Rev., 50(1): 59-87, 1998; Lisczewski, M K and Atkinson, J P, in The Human Complement System in Health and Disease, Volanakis, J E and Frank, M M, eds., Dekker, N.Y., pp. 149-66, 1998; Kuby Immunology, 2000; Paul, W. E., Fundamental Immunology, Lippincott Williams & Wilkins; 5^{th} ed., 2003; and Walport M J., Complement. First of two parts. N Engl J Med., 344(14):1058-66, 2001.

The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils.

The alternative pathway is initiated by, e.g., microbial surfaces and various complex polysaccharides. In this pathway, C3b, resulting from cleavage of C3, which occurs spontaneously at a low level, binds to targets, e.g., on cell surfaces and forms a complex with factor B, which is later cleaved by factor D, resulting in a C3 convertase. Cleavage of C3 and binding of another molecule of C3b to the C3 convertase gives rise to a C5 convertase. C3 and C5 convertases of this pathway are regulated by CR1, DAF, MCP, and fH. The mode of action of these proteins involves either decay accelerating activity (i.e., ability to dissociate convertases), ability to serve as cofactors in the degradation of C3b or C4b by factor I, or both.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains. These domains, consisting of approximately 50-70 amino acids, typically about 60 amino acids, are characterized by a conserved motif that includes four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4bp). CD59 is a membrane-bound complement regulatory protein unrelated structurally to the CCPs.

Complement Inhibition as a Therapeutic Approach in Trauma

The present invention provides the recognition that early intervention by administering a complement inhibitor at least once in the immediate post-trauma period (defined herein as within 24 hours following occurrence of a traumatic injury) will reduce morbidity and mortality in trauma patients. Furthermore, such therapy may be of use following the immediate post-trauma period as well. The invention provides a method of treating an individual who has suffered a traumatic injury comprising administering a complement inhibitor to the individual. In certain embodiments of the invention the complement inhibitor is administered within 24 hours following occurrence of the injury, e.g., within 1 hour following the occurrence of the injury. In some embodiments the complement inhibitor is administered essentially immediately following injury (i.e., within 5 minutes or less following injury). In some embodiments the complement inhibitor is administered at least 5 minutes following injury but within the first 30 minutes following injury, or in some embodiments within the first 1 hour following injury. In some embodiments the complement inhibitor is administered at least 5 minutes following injury but within 2, 3, 4, 6, 8, or 12 hours following injury. In some embodiments the complement inhibitor is administered at least once within any of the afore-mentioned time intervals and at least once following the time interval (e.g., at least once more than 24 hours following injury). In some embodiments a sufficient dose (or multiple doses) of complement inhibitor is administered to reduce complement activation (e.g., as measured by the level of C3 split products or any other method useful for measuring complement activation) in the trauma victim to within no more than 1, 2, or 5 times the average level in individuals who have not suffered trauma.

In certain embodiments of the invention the complement inhibitor inhibits all three major complement activation pathways. In certain embodiments of the invention the complement inhibitor inhibits activation of C3 or deactivates C3 or C5 convertases. In certain embodiments of the invention the complement inhibitor is a compstatin analog. In some embodiments the complement inhibitor is a C5a receptor antagonist. In some embodiments the complement inhibitor is soluble CR1 or a derivative such as TP20 (Avant Immunotherapeutics). In some embodiments the complement inhibitor is an anti-C3 or anti-C5 antibody or aptamer.

In some embodiments of the invention the complement inhibitor is a multimodal complement inhibitor, by which is meant a complement inhibitor that interferes with multiple steps in one or more complement activation pathways. In some embodiments of the invention the complement inhibitor is a bifunctional complement inhibitor comprising a first moiety that inhibits a first complement component and a second moiety that inhibits a receptor for a second complement component. In some embodiments of the invention the first moiety inhibits C3 and a second moiety that inhibits the C5a receptor.

The invention further provides a method of treating a trauma patient comprising: administering first and second complement inhibitors to the patient, wherein the first complement inhibitor inhibits C3 and the second complement inhibitor inhibits C5aR. In certain embodiments of the invention the complement inhibitors are administered as a single composition. The following sections describe strategies for patient selection, complement inhibitors of use in the inventive methods, and other aspects of the invention.

Diagnostic Information and Patient Selection

One aspect of the invention is methods of providing diagnostic information relevant to trauma outcome. For example, the invention provides a method of identifying a trauma patient as being at increased risk of having a poor outcome following trauma, the method comprising: determining the genotype of a trauma victim with respect to one or more polymorphisms associated with poor outcome following trauma, wherein said polymorphism is in or near a complement-related gene. The invention provides a method of identifying a trauma patient as being at increased risk of having a poor outcome following trauma, the method comprising: determining the genotype of a trauma victim with respect to one or more polymorphisms associated with poor outcome following trauma, wherein said polymorphism is in or near a complement-related gene and wherein at least one variant of said polymorphism is associated with increased risk of developing a complement-related disorder other than trauma.

One aspect of the invention relates to methods of selecting individuals that are suitable candidates for therapy with a complement inhibitor following traumatic injury. Such individuals include patients that have an increased likelihood of benefiting from administration of a complement inhibitor relative to other members of the general population having different characteristic(s). One such method comprises performing an anatomic and/or physiological assessment of injury severity in a trauma patient, wherein the trauma patient is selected as a candidate for therapy with a complement inhibitor if the injury severity is above a predetermined threshold. A second method comprises determining the genotype of the trauma patient with respect to a polymorphism, wherein at least one polymorphic variant of the polymorphism is associated with a poor outcome following trauma. In some embodiments the method comprises determining the genotype of the patient with respect to a gene, wherein at least two alleles of the gene exist in the population, and wherein at least one allele is associated with a poor outcome following trauma.

In some embodiments the methods comprise determining the genotype with respect to multiple such polymorphisms (which may be in or near the same or different genes) and/or multiple such genes. The genotype could be determined as a routine part of medical care and the information maintained in a database. The genotype may have been determined by a third party, such as an organization offering a genotyping service to the general public. The information, regardless of where or how maintained, would be accessed as appropriate if the individual suffered a traumatic injury. The genotype could additionally or alternately be determined after traumatic injury. Thus it will be appreciated that “determining” as used herein encompasses performing an analysis of a sample from the subject following trauma and thereby obtaining the genotype or accessing results that were initially determined prior to the trauma. Optionally, complement inhibitor therapy is administered based at least in part on the result. The invention thus encompasses a method comprising: (i) determining the genotype of a trauma victim with respect to one or more polymorphisms associated with poor outcome following trauma; (ii) making a treatment decision regarding administration of complement inhibitor therapy based at least in part on the genotype; and (iii) optionally administering one or more complement inhibitors to the trauma victim.

It will be appreciated that the genotype information used in the methods of the invention can be provided in any of a variety of formats. It can be provided with respect to one or both chromosomes carrying the polymorphic position or gene. For example, the genotype can comprise determining whether an individual is heterozygous or homozygous for a risk allele. It will be appreciated that the information need not be provided in terms of the nucleotide sequence itself For example it is typical to assign identifiers such as +/−, A, a, etc., to different alleles for descriptive purposes, provided that the locus referred to is provided or known.

A third method comprises measuring the level and/or activity of at least one complement-related protein, or any marker of complement activity (e.g., the C3a/C3 ratio) in an individual under “normal” conditions (i.e., when the individual has not recently suffered a traumatic injury). Individuals who have an elevated (relative to average members of the population) level of a complement protein (other than a CCP or other complement regulatory protein), a decreased level of a CCP or other complement regulatory protein, and/or an increased level of a marker of complement activity are at increased risk of poor outcome following trauma and are suitable candidates for complement inhibitor therapy in the immediate post-trauma period.

A fourth method comprises measuring the level and/or activity, and/or degree of depletion of at least one complement-related protein, or any marker of complement activity or activation (e.g., the C3a/C3 ratio) in an individual and/or the level, activity, and/or degree of depletion of an endogenous CCP or other complement regulatory protein during the immediate post-trauma period. In certain embodiments of the invention, individuals with an increased activity or depletion of at least one complement-related protein (other than a CCP or other complement regulatory protein), or an increased level of any marker of complement activity or activation (e.g., the C3a/C3 ratio) or a decreased level of a CCP are at increased risk of poor outcome following trauma and are suitable candidates for complement inhibitor therapy. In some embodiments, complement inhibitor therapy is used to maintain or reestablish a desired level of a biomarker, e.g., a biomarker that reflects complement activation. For example, in some embodiments, a complement inhibitor, e.g., a compstatin analog, is administered so as to bring or maintain the C3a/C3 ratio within a desired range or below a desired value. Typically, the desired range or value would be one that more closely resembles a normal range or value (e.g., a range or value found in healthy individuals who have not recently suffered severe trauma) than the range or value found in individuals who have recently suffered severe trauma. In exemplary embodiments, a complement inhibitor, e.g., a compstatin analog, is administered so as to bring or maintain the C3a/C3 ratio to within approximately 1, 1.2, 1.5, 2, 2.5, 3, 4, or 5 times the upper limit of the normal range. The effect of altered level of a CCP-like protein or other protein related in sequence to a complement regulatory protein will depend on factors such as whether the protein has complement controlling activity or rather has the effect of increasing complement activity by competing with a CCP or other complement regulatory protein.

It is envisioned that emergency response vehicles would be equipped with kits for rapidly determining genotype and/or making the aforementioned measurements of complement-related protein level and/or activity. Such kits are an aspect of the invention. It is also envisioned that genotype may be assessed while the patient is in the hospital. The invention provides a method comprising determining the genotype of a trauma patient with respect to one or more complement-related genes within X hours or less after the patient has suffered an injury, where “X” represents a number. In some embodiments of the invention X is 6. In some embodiments of the invention X is 12. In some embodiments of the invention X is 24. In some embodiments of the invention X is 48.

In some embodiments, selecting an appropriate individual to receive a complement inhibitor following traumatic injury involves assessing the severity of the injury using a trauma score. For purposes of the present invention, a scoring system that provides an indication of the extent of injury of a trauma victim is referred to as a trauma score (TS), it being understood that any of a variety of established injury scoring systems, or scoring systems developed for purposes of the present invention, may be used.

A number of scoring systems have been developed to provide a quantitative indicator of injury severity. The Revised Trauma Score (RTS) is a physiological scoring system, with high inter-rater reliability and demonstrated accurracy in predicting death. It is scored from the first set of data obtained on the patient, and consists of Glasgow Coma Scale, systolic blood pressure and respiratory rate (Champion H R et al, “A Revision of the Trauma Score”, J Trauma 29:623-629, 1989; Champion H R et al, “Trauma Score”, Crit Care Med 9:672-676, 1981). Values for the RTS are computed using the formula RTS=0.9368 GCS+0.7326 SBP+0.2908 RR and are typically in the range 0 to 7.8408. The Injury Severity Score (ISS) is an anatomical scoring system that provides an overall score for patients with multiple injuries Baker S P et al, “The Injury Severity Score: a method for describing patients with multiple injuries and evaluating emergency care”, J Trauma 14:187-196;1974. Each injury is assigned an Abbreviated Injury Scale (AIS) score and is allocated to one of six body regions (Head, Face, Chest, Abdomen, Extremities (including Pelvis), External). Only the highest AIS score in each body region is used. The 3 most severely injured body regions have their score squared and added together to produce the ISS score. The ISS score ranges from 0 to 75. If an injury is assigned an AIS of 6 (unsurvivable injury), the ISS score is automatically assigned to 75. The ISS score is the most widely used anatomical scoring system and correlates linearly with mortality, morbidity, hospital stay and other measures of severity. In some embodiments of the invention the complement inhibitor is administered to an individual having an ISS of at least 15. In some embodiments the complement inhibitor is administered to an individual having an ISS of at least 9. In some embodiments the individual has an RTS of at least 4.

The invention provides a method of treating an individual who has suffered a traumatic injury comprising steps of: (a) assessing the severity of the traumatic injury within 24 hours or less of injury; and (b) administering a complement inhibitor to the individual if the severity of the injury is above a predetermined level. In certain embodiments step (a) comprises determining a trauma score. In some embodiments the severity is assessed prior to the individual arriving at a health care facility such as a hospital. The assessment can take place at the location where the injury occurred or in a transport vehicle such as an ambulance or helicopter.

The invention further provides methods of selecting individuals that are suitable candidates for therapy with a complement inhibitor following traumatic injury, based at least in part on the genotype of the individual. Such individuals are more likely to benefit from administration of a complement inhibitor following traumatic injury than at least some individuals having different genotypic characteristics and may thus be particularly suitable candidates for complement inhibitor therapy. The invention encompasses the recognition that certain individuals are at increased risk of having a poor outcome following trauma as a consequence of their genome containing particular nucleotides at one or more polymorphic sites in the genome, wherein the polymorphic site is located in or near a complement-related gene. “Poor outcome” as used herein refers to any of a number of undesirable complications, sequelae, or events following traumatic injury that are not directly attributable to blood loss or direct tissue damage from the injury and typically manifest at least 24 hours following the injury. Poor outcomes include, e.g., (i) death; (ii) development of ARDS, severe SIRS (e.g., SIRS score of 4), MODS and/or MOF or any combination thereof; (iii) sepsis; and (iv) any combination of (i), (ii), and (iii), optionally excluding development of severe SIRS. A “favorable outcome” refers to an outcome other than a poor outcome. In some embodiments, a “favorable outcome” is an outcome other than a poor outcome under circumstances in which a poor outcome is expected or considered more likely than not to occur. In some embodiments, a patient who recovers from trauma without experiencing ARDS, MODS, MOF, or sepsis is considered to have a favorable outcome. In some embodiments, a patient who recovers from trauma without experiencing ARDS, severe SIRS, MODS, MOF, or sepsis is considered to have a favorable outcome.

Without wishing to be bound by theory, individuals having different polymorphic variants of a complement-related gene experience differences in complement activation, or differences in activity of complement regulatory protein(s), following traumatic injury, and the resulting differences in level of complement activity and/or complement activation (relative to that which would be expected to occur in otherwise similar individuals not having the polymorphic variant) contributes to the poor outcome or to the favorable outcome.

In certain embodiments of the invention the polymorphic variant associated with increased risk of poor trauma outcome is a variant of a gene, wherein certain polymorphic variants of the gene are known or strongly suspected (e.g., have been demonstrated in multiple genetic association studies) to be associated with increased susceptibility to a complement-mediated disorder or is located near such a gene. “Increased susceptibility to a disorder” refers to an increased risk of developing the disorder and/or an increased risk of developing a severe or rapidly progressive form of the disorder relative to individuals having the disorder but not harboring the polymorphic variant. For example, an individual has increased susceptibility to a disorder if the individual is more likely to develop the disorder than a comparable individual having a different genotype with respect to one or more alleles, wherein the comparable individual is otherwise similar with respect to other non-genetic risk factor(s) for the disorder (e.g., smoking history, diet) and/or standard demographic parameters such as age (e.g., up to 5 years younger or older), sex, and/or race.

It should be noted that the effect of a polymorphism on risk of developing a complement-mediated disorder may differ qualitatively and/or quantitatively from the effect of such polymorphism on risk of poor outcome following trauma For example, in some embodiments of the invention an allele associated with increased risk of developing a complement-mediated disorder is associated with increased risk of poor outcome following trauma, while in other embodiments of the invention an allele associated with increased risk of developing a complement-mediated disorder is associated with decreased risk of poor outcome following trauma. Likewise, in some embodiments of the invention an allele associated with decreased risk of developing a complement-mediated disorder is associated with decreased risk of poor outcome following trauma, while in other embodiments of the inventionan an allele associated with decreased risk of developing a complement-mediated disorder is associated with increased risk of poor outcome following trauma.

In one embodiment the complement-mediated disorder is AMD. The polymorphic variant assessed in the method of the invention may be the same as the polymorphic variant that is associated with increased susceptibility to the complement-mediated disorder, or presence of a variant associated with decreased susceptibility may be assessed. Furthermore, it is recognized that additional polymorphic variants are likely to exist in or near such genes (“near” refers to, e.g., within 25 kB, within 50 kB, within 100 kB, or within 150 kB of the known or predicted 5′ or 3′ end of the gene, in various embodiments of the invention), and such variants are also of use in the present invention.

Genetic studies have demonstrated association between certain alleles of genes encoding various complement-related proteins and increased susceptibility to developing AMD and/or increased likelihood of developing a severe form of AMD. An allele that is associated with increased likelihood of developing a disorder or condition and/or increased likelihood of developing a severe form of the disorder or condition or having a poor outcome from the disorder or condition is referred to herein as a “risk allele” for that disorder or condition, and the gene is referred to as a “risk modifier” for the disorder or condition. The risk allele may, for example, be the allele found at higher frequency in cases than controls in a case control study. For example, an “AMD risk allele” is an allele that is associated with increased likelihood of developing AMD and/or increased likelihood of developing a severe form of AMD.

Certain risk alleles of interest herein are alleles of the gene that encodes complement factor H (CFH), wherein the alleles contain a polymorphism resulting in a CFH isoform that contains His rather than Tyr at position 402 (Tyr402His polymorphism), e.g., resulting from a T to C substitution at nucleotide 1277 in exon 9. Without wishing to be bound by any theory, the Tyr402His variant of CFH may be less effective at controlling complement activation and/or may have altered tissue localization adversely affecting its complement control ability. Subsequent studies have found that other CFH isoforms are tightly associated with AMD risk (Klein, R. J. et al. Complement Factor H Polymorphism in Age-Related Macular Degeneration. Science (2005); Edwards, A. O. et al. Complement Factor H Polymorphism and Age-Related Macular Degeneration. Science (2005); Haines, J. L. et al. Complement Factor H Variant Increases the Risk of Age-Related Macular Degeneration. Science (2005).

Furthermore, variants of the genes that encode complement proteins C2, C3, factor B, C7 and MBL-2 have also been associated with AMD risk (Gold, B. et al. Variation in factor B (BF) and complement component 2 (C2) genes is associated with age-related macular degeneration. Nat. Genet. 38, 458-462 (2006); Dinu, V. et al. Evidence for Association between Multiple Complement Pathway Genes and AMD. Genet. Epidem. 31, 224-237 (2007) Yates, J. R. W., Complement C3 Variant and the Risk of Age-Related Macular Degeneration, N. Engl. J. Med., 357: 19-27, 2007). Certain alleles of such genes (“protective alleles”) have been associated with a reduced risk of developing AMD relative to other alleles. It will be appreciated that in such instances, having the non-protective allele(s) may be considered to increase risk of developing AMD and, in accordance with certain embodiments of the present invention, the risk of poor outcome following trauma. In other instances, having the non-protective allele(s) with respect to AMD decreases the risk of poor outcome following trauma.

In some embodiments the complement-mediated disorder is hemolytic uremic syndrome (HUS). Genetic abnormalities in complement regulatory proteins, including complement factor H (CFH), membrane cofactor protein (MCP), and complement factor I (FI), and in CFHR1 and CFHR3, have been reported in 30%, 10%, and 5% of patients with atypical HUS, respectively (see, e.g., Caprioli J, et al., Genetics of HUS: The impact of MCP, CFH and IF mutations on clinical presentation, response to treatment and outcome. Blood 108: 1267-1279, 2006; Richards A, et al., Factor H mutations in haemolytic uremic syndrome cluster in exons 18-20, a domain important for host cell recognition. Am J Hum Genet 68: 485-490, 2001; Fremeaux-Bacchi V, et al., Genetic and functional analyses of membrane cofactor protein (CD46) mutations in atypical haemolytic uremic syndrome. J Am Soc Nephrol 17: 2017-2025, 2006; Zipfel, P., et al., PLOS Genetics, Vol. 3(3): 387-392, e41, March 2007. Some mutations, such as G3587T, which introduces a stop codon at position 1172, eliminate one or more of the C-terminal short concensus repeats. This mutation severely affected recognition functions (i.e., binding to heparin, C3b, C3d, and the surface of endothelial cells). On the surface of endothelial cells, the mutant factor H protein showed severely reduced regulatory activities and lacked protective functions, leads to defective complement control on cell surfaces (Heinen, S., et al., Hemolytic uremic syndrome: a factor H mutation (E1172Stop) causes defective complement control at the surface of endothelial cells, Am Soc Nephrol. (2007)18(2):506-14, 2007).

CFI is a two-chain serine protease in which the light chain carries the catalytic domain. CFI downregulates the alternative and classical complement pathways by cleaving the α chains of C3b and C4b in the presence of cofactor proteins. Mutations I322T, D501N and D506V reside in the serine protease domain of CFI and result in secreted proteins that lack C3b and C4b cofactor activity. The de1TTCAC (1446-1450) mutant leads to a protein that is not secreted. The R299W mutant lies in a region of the CFI heavy chain of no known function (Kavanagh, D., Characterization of mutations in complement factor I (CFI) associated with hemolytic uremic syndrome; Mol Immunol; 45(1), pp. 95-195, 2008). Other CFI mutations of interest are found in the CFI mutation database (CFIbase) at the website having URL: bioinf.uta.fi/CFIbase/?content=pub/IDbases.

At least 70 CFH mutations have been reported in patients with aHUS (listed in the database at URL www.FH-HUS.org, The Factor H-Associated HUS Mutation Database). Most of these mutations are heterozygous missense mutations that cluster in the exons encoding SCRs 19 and 20. Functional studies on the mutant proteins have shown reduced C3b and heparin binding that results in impaired control of complement activation on the endothelial cell surface. These mutations include two nucleotide changes, c.3572C>T and c.3590T>C, that in some patients occur in combination. Also reported in the afore-mentioned database are 12 Factor I mutations and 25 MCP mutations linked with HUS and 6 mutations within CFH that are associated with membranous proliferative glomerulonephritis (MPGN), another complement-mediated disorder. These mutations are thought to lead to inability to appropriately control the complement cascade at sites of cell injury and tissue damage such as that occurring in trauma.

The invention encompasses assessing the genotype of an individual with respect to any of the afore-mentioned mutations and polymorphisms associated with HUS or MPGN, wherein an individual having one or more such mutations or polymorphisms is at increased or decreased risk of poor outcome following trauma. It will be appreciated that these mutations and polymorphisms are merely exemplary. The invention encompasses assessing the genotype of an individual with respect to any mutation or polymorphism that has been demonstrated to result in (or is genetically associated with), decreased ability to control the complement cascade at sites of cell damage and/or tissue injury.

A number of other genetic loci have been identified wherein variations influence a subject's risk of developing AMD. Certain of these loci do not at present have a known role in the complement system or in complement-mediated disorders independent of their association with AMD. For example, variations within loci on human chromosome 10, e.g., loci located within 10q26 such as the PLEKHA1 or LOC387715/ARMS2/HTRA1 loci, are associated with altered, e.g., increased risk of AMD. See, e.g., U.S. Pub. No. 20060281120 and WO/2006/133295, entitled SUSCEPTIBILITY GENES FOR AGE-RELATED MACULOPATHY (ARM) ON CHROMOSOME 10q26. The A69S (alanine to serine substitution) variant of LOC387715/ARMS2 (rs10490924) is one such variant associated with increased risk of AMD. The allele having a “T” at the polymorphic position is associated with increased risk of AMD. The present invention encompasses the recognition that such variations may also be associated with altered (increased or decreased) risk of poor outcome following trauma. Accordingly, the invention encompasses assessing the genotype of an individual with respect to polymorphisms located in the PLEKHA1 or LOC387715/ARMS2/HTRA1 loci, e.g., the A69S variant of LOC387715/ARMS2 (minor allele or SNP rs10490924).

The present invention encompasses assessing the genotype of a trauma patient with respect to any of the genes and/or polymorphisms described in the afore-mentioned studies, or with respect to any other gene(s) encoding a complement-related protein or gene in which mutation or variation has been linked to a complement-mediated disorder (see above) and selecting the trauma patient as a suitable candidate for administration of a complement inhibitor based at least in part on the results of said assessment.

In some embodiments of the invention, the particular complement inhibitor to be administered is selected based at least in part on the results of said assessment. For example, if the individual harbors an allele that is associated with increased alternative pathway activity, e.g., an allele that results in overactivity of the alternative pathway, a complement inhibitor specific for the alternative pathway may be administered. If the individual harbors an allele that results in diminished activity of a protein that downregulates complement activity, the “normal” variant of that protein may be administered to the individual. For example, CFH or CFI could be administered to individuals having alleles associated with decreased CFH or CFI activity.

Exemplary SNPs of interest herein are listed below:
rs1061170
rs2274700
rs1410996
rs7535263
rs10801559
rs3766405
rs10754199
rs1329428
rs10922104
rs1887973
rs10922105
rs4658046
rs10465586
rs3753396
rs402056
rs7529589
rs7514261
rs10922102
rs10922103
rs800290
rs1061147
rs1048663
rs412852
rs11582939
rs1280514
rs2876849
rs930508
rs2250656
rs2230203
rs2230204
rs2287846
rs344542
rs2241393
rs344550
rs2277984
rs7033790
rs17611
rs7026551
rs3753394
rs800292 (I62V)
rs380390
rs3766404
rs529825 (IVS1)
rs203674

Other SNPs of interest include the following (names of genes containing the relevant polymorphism are indicated in parentheses):
rs1047286 (C3)
rs2230199 (C3)
rs120862610 (C5)
rs9332739 (C2)
rs547154 (C2)
rs4151667 (CFB)
rs641153 (CFB)
rs41015361 (C7)
rs33682798 (MBL2)
rs930508 (MBL2)
rs10490924 (LOC387715/ARMS2)
rs1045216 (PLEKHA1)

Yet other SNPs of interest are:
rs4146894
rs1882907
rs760336
rs763720
rs800292
rs1483883
rs1853886
rs11200638

In some embodiments, the polymorphism is one that results in versions of the C3 protein that correspond to the electrophoretic variants C3S (slow) and C3F (fast). For example, one such polymorphism is the SNP rs2230199 (Arg80Gly), wherein the allele encoding a C3 protein having a Gly at position 80 corresponds to the F variant and is the risk allele for AMD. Without being bound by any theory, and without limiting the invention in any way, the C3F variant of C3 (e.g., a variant having a Gly at position 80) may be associated with increased or decreased risk of poor outcome following trauma.

In some embodiments, at least two SNPs are evaluated, wherein a first SNP is in the CFH gene and a second SNP is in a second locus or gene selected from the group consisting of C3, CFB, C7, LOC387715/ARMS2/HTRA1, and PLEKHA1. In some embodiments, the second gene is the C3 gene. In some embodiments the gene or locus is LOC387715/ARMS2. In some embodiments the second gene or locus is PLEKHA1.

In some embodiments, at least one SNP is selected from: rs1061170, rs1047286, rs2230199, rs120862610, rs9332739, rs547154, rs4151667, rs641153, rs41015361, rs33682798, rs10490924, and rs1045216. In some embodiments at least two SNPs are selected from: rs1061170, rs1047286, rs2230199, rs120862610, rs9332739, rs547154, rs4151667, rs641153, rs41015361, rs33682798, rs10490924, and rs1045216. In some embodiments at least three SNPs are selected from: rs1061170, rs1047286, rs2230199, rs120862610, rs9332739, rs547154, rs4151667, rs641153, rs41015361, rs33682798, rs10490924, and rs1045216.

In some embodiments, at least one SNP is selected from: rs1061170, rs2230199, rs10490924, rs641153, rs1047286, and rs9332739. In some embodiments at least one SNP is selected from: rs7033790, rs17611, rs120862610, rs547154, rs4151667, rs41015361, rs33682798, and rs1045216.

In some embodiments, at least one of the polymorphisms is in the CFB or C2 gene, for example the polymorphisms disclosed as being linked to protection from AMD in PCT/US2006/003904, PCT/US2006/003696, and/or PCT/US2007/061964. Without limitation, the method may comprise determining whether a subject has any one or more of the following: (i) A or G at rs641153 of the CFB gene, or R or Q at position 32 of the CFB protein; (ii) A or T at rs4151667 of the CFB gene, or L or H at position 9 of the CFB protein; (iii) G or T at rs547154 of the C2 gene or ; and (iv) C or G at rs9332739 of the C2 gene, or E or D at position 318 of the C2 protein.

Individuals having certain combinations of polymorphic variants may be at especially high risk of a poor outcome following trauma. In such instances the dose of complement inhibitor to be administered and/or the identity of the complement inhibitor and/or the duration over which complement inhibitor therapy is administered may be modified.

In some embodiments, the method comprises assessing an individual's genotype at the CBF or C2 locus and/or at the CFH locus with respect to whether the individual is: (i) heterozygous for the R32Q polymorphism in CFB; (ii) heterozygous for the L9H polymorphism in CFB; (iii) heterozygous for the IVS 10 polymorphism in C2; (iv) heterozygous for the E318D polymorphism in C2; (v) homozygous for the delTT polymorphism in CFH; and/or (vi) homozygous for the R150R polymorphism in CFB and, optionally, homozygous for Y402H in CFH.

It will be appreciated that polymorphisms, e.g., SNPs, may be in linkage disequilibrium (LD) with other polymorphisms, e.g., other SNPs, located on the same chromosome. Such SNPs may be present in haplotypes. For example, some SNPs may be linked over distances of up to 100 kB or even over 150 kB or more (Reich, D. E., et al., Nature, 411, 199-204, 2001). Thus in some embodiments the methods of the invention comprise determining whether an individual has a haplotype that comprises at least one polymorphic variant of a polymorphism associated with increased risk of poor outcome following trauma, wherein said polymorphism is in a complement-related gene. In some embodiments the haplotype comprises the Tyr402His coding variant of the CFH gene. The individual may be heterozygous for the haplotype. In some embodiments the haplotype is a CFH haplotype that does not comprise the Tyr402His coding variant (see, e.g., Li, et al., supra). The individual may be homozygous for the haplotype. In some embodiments the SNP is associated with a particularly high risk of developing wet AMD.

Polymorphisms of interest can be single nucleotide polymorphisms (SNPs), deletions, insertions, or any kind of genetic variation. Typically such variations are present at a level of at least 1% in the population of interest, but less common variations, and mutations, are of interest as well. Polymorphisms (e.g., SNPs) that are linked to the specific polymorphisms (e.g., SNPs) disclosed herein are of use in the methods of the present invention. As in the case of certain polymorphisms dislosed above, the frequencies of certain variant forms of a linked polymorphism of use in the methods of the invention differ between those individuals who had a poor outcome following trauma and those individuals who did not have a poor outcome following trauma and/or between those individuals who have or are susceptible to AMD or another complement-mediated disorder, and those individuals who do not have the disorder or increased susceptibility to the disorder. Therefore, one or more variant forms of a linked polymorphism may be associated with poor outcome following trauma and is of use in method of the present invention. In some embodiments the polymorphism is in strong linkage disequilibrium with any of the afore-mentioned polymorphisms associated with risk of a complement-mediated disorder. A variety of metrics are known in the art to evaluate the extent to which any two alleles are in linkage disequilibrium (LD). Suitable metrics include D′, r2, and others (see, e.g., Hedrick, P. W., Genetics, 117(2):331-41, 1987). As used herein, “strong LD” is said to exist if D′>0.8.

A large number of SNPs and their chromosomal locations are known in the art and are publicly available in databases such as dbSNP, provided by the National Center for Biotechnology Information (NCBI) (available at http://www.ncbi.nlm.nih.gov/projects/SNP/), the International HapMap Project (available at www.hapmap.org), etc. These databases provide a wide variety of information including, e.g., the identity of the nucleotide at each polymorphic position, whether it is a major or minor allele, the sequence surrounding each polymorphic position, chromosome and chromosomal location, gene names and identifiers for SNPs that lie within genes, biological notation, if available, etc. Thus one of ordinary skill in the art can readily identify SNPs in or near particular genes of interest, can readily determine the identity of the possible nucleotides at the polymorphic site and the surrounding sequence, and can readily design probes, primers, etc., to detect such SNPs and/or genotype individuals with respect to such SNPs. In addition, considerable information regarding numerous SNPs is available at the Affymetrix NetAffx™ Analysis Center (www.affymetrix.com/analysis/index.affx). The invention also encompasses identification of new SNPs in or linked to genes encoding complement-related proteins and their use in the methods described herein.

The methods of the invention are of use to provide diagnostic information relevant to a trauma patient. As used herein, “diagnostic information” includes information that is useful in determining whether a patient is susceptible to having a poor outcome following trauma and/or in classifying the patient into a category having significance with respect to prognosis or likelihood of response to treatment (either treatment in general or any particular treatment, e.g., complement inhibitor therapy).

In some embodiments, the identity and/or dose of a complement inhibitor can be selected to restore the individual to a state of “normal” activity with respect to a complement control protein, wherein “normal” in this context refers to the level of activity of such complement control protein found in a non-injured individual, e.g., a non-injured individual who does not harbor a polymorphic variant of said complement control protein that predisposes the individual to a complement-mediated disorder and/or to a poor outcome following trauma.

It will be appreciated that the present disclosure should not be understood as limiting the therapeutic aspects of the invention to certain individuals (e.g., individuals having a genotype associated with increased risk of poor outcome following trauma) or as implying that other individuals (e.g., individuals not having a genotype associated with increased risk of poor outcome following trauma) should not be treated with a complement inhibitor following traumatic injury. Furthermore, other methods of selecting individuals having increased likelihood of benefiting from complement inhibitor therapy following trauma may exist or may be discovered in the future. Notwithstanding the foregoing, correlating the genotype of an individual with respect to one or more polymorphisms in or near a complement-related gene with outcome following trauma may be of use to identify individuals who are unlikely to benefit from complement inhibitor therapy and/or who in whom such therapy might be contra-indicated. Such methods are an additional aspect of the invention.

Methods of Assessing Genotype

The genotype of the individual can be determined using any of a variety of methods. The particular method employed is not critical to the present invention and need not be described here in detail, such methods being well known in the art. The methods typically utilize a biological sample obtained from the individual, wherein the sample comprises nucleic acids and/or proteins. As used herein, a “biological sample” refers to any of the following: a cell or cells, a portion of tissue, a body fluid such as blood, urine, saliva, cerebrospinal fluid, etc. The term “biological sample” also includes any material derived by processing a biological sample as previously defined, e.g., by isolating or purifying DNA, RNA, and/or protein, from the sample, by subjecting the sample or a portion thereof to amplification, restriction enzyme digestion, etc. Typically a blood or tissue sample is used. Methods can involve testing the individual's DNA to determine whether the DNA comprises an allele or polymorphism of interest. RNA can also be used, if the polymorphism of interest lies in a portion of the gene that is transcribed. In some embodiments the methods involve determining the identity of a particular nucleotide, wherein polymorphism(s) at the position of such nucleotide are associated with increased or decreased risk of poor outcome following trauma and/or increased or decreased susceptibility to AMD or another complement-mediated disorder.

Methods for performing such tests are well known in the art and include, e.g., isolating the DNA or RNA, optionally amplifying it (e.g., using the polymerase chain reaction (PCR) or reverse transcriptase-PCR), and performing a variety of methods such as allele-specific primer extension, allele-specific hybridization, restriction enzyme digestion, sequencing, etc. In some embodiments genotyping is performed using a microarray, or “chip”. In some embodiments genotyping is performed using a bead-based assay such as the Luminex platform. Other methods of use include oligonucleotide ligation assays (U.S. Pat. Nos. 5,185,243, 5,679,524 and 5,573,907), cleavage assays, heteroduplex tracking analysis (HTA) assays, etc. Examples include the Taqman® assay, Applied Biosystems (U.S. Pat. No. 5,723,591). Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader® assays (Third Wave Technologies), described in Eis, P. S. et al., Nat. Biotechnol. 19:673, 2001, can also be used. Assays based on molecular beacons (U.S. Pat. Nos. 6,277,607; 6,150,097; 6,037,130) or fluorescence energy transfer (FRET) may be used. U.S. Pub. No. 20050069908 and references therein describe a variety of other methods that can be used for the detection of nucleic acids. U.S. Pat. Nos. 5,854,033, 6,143,495, and 6,239,150 describe compositions and a method for amplification of and multiplex detection of molecules of interest involving rolling circle replication. The method is useful for simultaneously detecting multiple specific nucleic acids in a sample. Optionally the nucleic acids are sequenced. U.S. Pub. No. 20050026180 describes methods for multiplexing nucleic acid reactions, including amplification, detection and genotyping, which can be adapted for determining the sequence at specific locations of interest for purposes of determining whether an individual has a genotype associated with increased risk of poor outcome following trauma.

In summary, and without limitation, suitable methods include hybridization-based methods such as dynamic allele-specific hybridization, use of molecular beacons, SNP microarrays, enzyme-based methods such as those based on restriction fragment length polymorphism, PCR-based methods, methods employing flap endonuclease, primer extension, 5′-nuclease, oligonucleotide ligase assay, other post-amplification methods based on physical properties of DNA, single strand conformation polymorphism, temperature gradient gel electrophoresis, denaturing high performance liquid chromatography, and sequencing. High throughput sequencing is becoming more efficient at a rapid pace, and it is envisioned that sequencing may become routinely used for genotyping purposes. Methods based on pyrosequencing, in situ sequencing, bead-based sequencing (US2007087362), etc., are of use. See also PCT/US2006/029449 (W02007014338) for further information and related definitions.

Certain methods comprise measuring the expression level of the mRNA encoding the protein or the level of the protein itself. Such methods are of use when the allele of interest has an altered level of expression relative to other alleles. For example, a polymorphism in a regulatory region of a gene may result in increased or decreased expression. Alterations in mRNA expression can be assessed using hybridization-based approaches (e.g., microarray, Northern blot, quantitative RT-PCR, etc.). Alterations in protein expression can be assessed using, e.g., protein microarrays, Western blot, mass spectrometry, etc.

Other methods of determining the genotype are based on assessing one or more characteristics of the protein encoded by the gene of interest other than expression level. For example, such methods can comprise detecting a variant form of a protein based on, e.g., differences in electrophoretic mobility, differential binding to a target protein or ligand, immunological methods employing antibodies or other compounds (e.g., aptamers) capable of selectively binding to one or more variants of the protein, mass spectrometry, etc. For purposes of the present invention, methods based on assessing mRNA or protein expression level and/or a characteristic of the protein are referred to as phenotype-based methods of determining the genotype.

As noted above, in certain embodiments of the invention genotype is determined shortly before administering the complement inhibitor, e.g., after the individual has experienced a traumatic injury. In certain embodiments of the invention the genotype will have been determined prior to occurrence of the injury. The invention thus provides a method of treating an individual who has suffered a traumatic injury comprising administering a complement inhibitor to the individual, wherein the individual is one whose genotype with respect to a polymorphism located in or near a gene that encodes a complement-related protein has been determined prior to administering the complement inhibitor. In certain embodiments of the method, the polymorphism is associated with risk of poor outcome following trauma. The invention further provides a method of treating an individual who has suffered a traumatic injury comprising (i) determining the genotype of the individual with respect to a polymorphism located in or near a gene that encodes a complement-related protein; and (ii) administering a complement inhibitor to the individual based at least in part on the genotype. In certain embodiments of the invention the polymorphism is one that has been associated with risk of poor outcome following trauma, i.e., at least one allele of the polymorphism is a trauma risk allele. In certain embodiments of the invention the polymorphism is one that has been associated with risk of developing a complement-mediated disorder or risk of developing a severe form of the disorder, i.e., at least one allele of the polymorphism is a risk allele for the disorder. In certain embodiments of the invention the disorder is AMD.

Identification of Risk Alleles and SNPs

The present application discloses a number of polymorphisms and risk alleles of use in the methods for providing diagnostic information, selecting a patient, and/or making a treatment decision such as selecting a therapeutic agent described herein. The invention also encompasses the recognition that additional polymorphisms and risk alleles of use are likely to exist. The invention provides methods to identify polymorphisms and risk alleles and to identify genes and genetic loci that harbor them. The invention further provides methods of identifying genetic variations that predispose, i.e., increase susceptibility of, an individual to having a poor outcome following trauma. Such methods include, e.g., association studies (e.g., whole genome association studies), linkage studies, etc. In some embodiments the methods focus on complement-related genes and identify risk alleles of such complement-related genes and/or of polymorphisms in or near such genes, wherein the polymorphism is associated with increased susceptibility to poor outcome following trauma (i.e., one or more alleles of the polymorphism is associated with increased susceptibility to poor outcome following trauma). The methods can involve, e.g., obtaining samples from a set of trauma patients that had a poor outcome and a set of trauma patients that did not have a poor outcome. The patients are genotyped with respect to polymorphisms known in the art to be located in or near such genes (e.g., within up to 100 kb or 150 kb of such genes. Correlations are sought between presence of particular alleles of one or more polymorphisms and poor outcome using methods known in the art (see, e.g., Dinu, supra). Methods of identifying such correlations are well known in the art and need not be described in detail here. In certain embodiments, presence of a particular allele at a frequency greater than expected by chance (e.g., p <0.05) in a group of patients with a poor outcome following trauma is indicative of the allele being a trauma risk allele. The methods may be practiced using polymorphisms disclosed herein or others known in the art or discovered in the future. It will be appreciated that the set of trauma patients analyzed can exclude or include trauma patients having certain characteristics. For example, the trauma patients may include individuals having an ISS within a predetermined range while excluding individuals having an ISS outside the range. Subpopulations such as males, females, individuals within a particular age range, or individuals having a particular type of injury (e.g., crush injury, penetrating trauma, head injury, burn), etc., may be analyzed.

In some embodiment the invention provides a method of identifying a polymorphism associated with outcome following trauma, the method comprising: (a) determining the genotype of a plurality of individuals that had a poor outcome following a traumatic injury with respect to one or more polymorphisms in or near a complement-related gene; (b) determining the genotype of a plurality of control individuals that suffered a traumatic injury of similar severity and did not have a poor outcome with respect to the one or more polymorphisms in or near the complement-related gene; and (c) determining correlations between the genotype of the individuals and occurrence of a poor outcome, wherein one or more significant correlation(s) identifies a polymorphism within or near the gene as being associated with outcome following trauma. In some embodiments, the method identifies one or more alleles of a polymorphism as being associated with increased risk of poor outcome following trauma.

The method may be performed using a collection of samples from trauma patients. For example, collections of between 100 and 2000 samples may be a convenient size to detect correlations of interest. Methods that comprise performing such an analysis, and results obtained therefrom, and methods of using such results, are aspects of this invention.

The invention provides a database comprising a list, table, or other appropriate form of presentation of genes, haplotypes, alleles, and/or polymorphisms stored on a computer-readable medium, wherein the contents of the database with respect to genes, haplotypes, alleles, and/or polymorphisms includes, and in some embodiments is largely (e.g., at least 75%) or entirely limited to, information relating to genes, haplotypes, alleles, and/or polymorphisms that have been identified as useful in performing genotyping to assess an individual's likelihood of having a poor outcome following trauma and/or for determining whether an individual is a suitable candidate for therapy with a complement inhibitor. The database can in the form of any assemblage or compendium of information and is optionally searchable. Optionally the information is selected at least in part to be useful with respect to one or more methods of the present invention. The database can include, e.g., information such as relative risk associated with particular genotypes, alleles, or haplotypes; allele frequencies. The information can be stored in any of a wide variety of formats. The database may include results of genotyping one or more individuals (e.g., trauma patients) with respect to one or more of the haplotypes, alleles, and/or polymorphisms described herein. The invention encompasses a method comprising the step of electronically sending or receiving information present in a database of the invention. The invention also encompasses a method comprising the step of electronically sending or receiving results of a genotyping test described herein. In some embodiments, the results are transmitted to an emergency response unit, e.g., an emergency response vehicle, field hospital, emergency room, etc. In some embodiments results are transmitted and/or received wirelessly. The database may be stored on a suitable computer-readable medium, e.g., a hard drive, compact disk, flash drive, etc. Further provided is a computer system containing memory that stores and/or accesses the database and a processor that processes information contained therein and, optionally, one or more input, output, or display devices.

The identified trauma risk alleles can be used to provide prognostic or other diagnostic information, guide therapeutic decisions, etc., as described herein. The identified trauma risk alleles can also be used to retrospectively or prospectively analyze the outcomes of patients who have particular characteristics or who have received particular treatments. For example, it would be of interest to determine whether particular treatments or interventions, e.g., administration of certain drugs, blood transfusion, mechanical ventilation, placement of a pulmonary artery catheter, etc., has a differential effect on patients having a trauma risk allele, e.g., whether such treatment or intervention is more or less likely to benefit a patient who is homozygous or heterozygous for the trauma risk allele relative to the likelihood of benefiting an individual who is not homozygous for the trauma risk allele or who is not heterozygous for the trauma risk allele. Such approaches are of use to identify subsets of patients that may be particularly likely to benefit from such treatment or intervention or who may be adversely affected by such treatment or intervention.

Reagents and Kits

The invention provides isolated nucleic acids whose sequence comprises a site in the human genome at which a polymorphism exists, wherein said polymorphism is associated with an increased risk of poor outcome following trauma. In some embodiments, the site is located in or near gene that encodes a complement-related protein. The sequence may be, e.g., between 8 and 100 nucleotides in length.

The invention further provides probes, primers, antibodies, and other reagents useful for detecting the alleles and polymorphisms (or protein isoforms encoded by variant alleles) described herein. Other reagents may include, e.g., buffers, enzymes, detection means, and other ancillary components useful for performing an assay. In certain embodiments the probes, primers, and other reagents are suitable for performing, e.g., any of the assays described above useful for genotyping an individual with respect to one or more polymorphisms in or near a complement-related gene. In certain embodiments a collection of probes, primers, antibodies, and/or reagents is provided. In certain embodiments the collection is especially adapted for predicting the susceptibility of an individual to a poor outcome following trauma. For example, the collection may be capable of genotyping with respect to a set of polymorphisms that is more informative with regard to risk of poor outcome following trauma than with regard to susceptibility to one or more complement-mediated disorders, e.g., AMD. In some embodiments, at least 50%, at least 75%, or at least 90% of the probes, primers, or antibodies in the collection are capable of use to detect polymorphisms or isoforms that are associated with increased or decreased susceptibility to poor outcome following trauma.

The probes, primers, etc., may be provided in the form of a kit. In some embodiments the kit is adapted for predicting the susceptibility of an individual to a poor outcome following trauma. In some embodiments the probes or primers are provided as an array, e.g., a microarray. In certain embodiments the probes or primers are linked to particles such as beads, e.g., magnetic particles such as magnetic beads.

Complement Inhibitors

Compstatin Analogs

Compstatin is a cyclic peptide that binds to complement component C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 41), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), and discussion below.

The invention encompasses the recognition that compstatin analogs possess unique and unexpected advantages as compared with other complement inhibitors for treatment of trauma. The relatively low molecular weight (˜1.6 kD) and various other properties of compstatin analogs facilitate their penetration into extravascular tissues and, in certain embodiments of the invention, potentially their ability to enter the CNS, allowing for therapeutic concentrations in target tissues.

Compstatin analogs may be acetylated or amidated, e.g., at the N-terminus and/or C-terminus For example, compstatin analogs may be acetylated at the N-terminus and amidated at the C-terminus Consistent with usage in the art, “compstatin” as used herein, and the activities of compstatin analogs described herein relative to that of compstatin, refer to compstatin amidated at the C-terminus (Mallik, 2005, supra).

Concatamers or multimers of compstatin or a complement inhibiting analog thereof are also of use in the present invention.

As used herein, the term “compstatin analog” includes compstatin and any complement inhibiting analog thereof The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain compstatin analogs and suitable assays are described in U.S. Pat. No. 6,319,897, WO2004/026328, Morikis, supra, Mallik, supra, and/or Katragadda 2006, supra. The assay may, for example, measure alternative pathway-mediated erythrocyte lysis or be an ELISA assay (see Examples 4 and 5). WO2004/026328, Morikis, supra, Mallik, supra, and Katragadda 2006, supra, among other references, describe compstatin analogs having higher activity than compstatin and methods for determining their ability to inhibit complement activation. The invention includes embodiments in which any one or more of the compstatin analogs or compositions described herein is used in any the methods of treatment described herein. 1⁰⁰¹⁵⁰1 The activity of a compstatin analog may be expressed in terms of its IC₅₀ (the concentration of the compound that inhibits complement activation by 50%), with a lower IC₅₀ indicating a higher activity as recognized in the art. The activity of a preferred compstatin analog for use in the present invention is at least as great as that of compstatin. It is noted that certain modifications known to reduce or eliminate complement inhibiting activity and may be explicitly excluded from any embodiment of the invention. The IC₅₀ of compstatin has been measured as 12 μM using an alternative pathway-mediated erythrocyte lysis assay (WO2004/026328). It will be appreciated that the precise ICₛₒ value measured for a given compstatin analog will vary with experimental conditions (e.g., the serum concentration used in the assay). Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments of the invention the activity of the compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments the activity is between 264 and 300, 300 and 350, 350 and 400, or 400 and 500 times as great as that of compstatin. The invention further contemplates compstatin analogs having activities between 500 and 1000 times that of compstatin.

The K_{d} of compstatin binding to C3 can be measured using isothermal titration calorimetry (Katragadda, et al., J. Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μmM and 0.1 μM, between 0.025 μM and 0.05 μM, between 0.015 μM and 0.025 μM, between 0.01 μM and 0.015 μM, or between 0.001 μM and 0.01μM. In certain embodiments the IC₅₀ of the compstatin analog is between about 0.2 μM and about 0.5 μM. In certain embodiments the IC₅₀ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH-W), 5-hydroxy-L-tryptophan (5OH-W), 6-hydroxy-L-tryptophan (6OH-W), 1-methyl-L-tryptophan (1MeW), 4-methyl-L-tryptophan (4MeW), 5-methyl-L-tryptophan (SMeW), 7-aza-L-tryptophan (7aW), α-methyl-L-tryptophan (αMeW), β-methyl-L-tryptophan (βMeW), N-methyl-L-tryptophan (NMeW), ornithine (orn), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components.

In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention X′aa is an analog of Trp that has increased hydrophobic character relative to Trp. For example, X′aa may be 1-methyltryptophan. In certain embodiments of the invention Xaa is an analog of Trp that has increased propensity to form hydrogen bonds relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments of the invention X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention Xaa is an analog of Trp that has increased propensity to form hydrogen bonds with C3 relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments X″aa is selected from analogs of Trp, aromatic amino acids, and aromatic amino acid analogs.

In certain preferred embodiments of the invention the peptide is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10-12 amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S—S) bond. In other embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:
Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and
   the two Cys residues are joined by a disulfide bond.

In other embodiments Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The invention further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the negative charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:
Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and
   the two Cys residues are joined by a disulfide bond.

Xaa1, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments of the invention Xaa1 is Ile and Xaa4 is L-Thr.

In certain embodiments of the invention Xaal is Ile, Xaa2* is Trp, and Xaa4 is L-Thr.

The invention further provides compstatin analogs of SEQ ID NO: 7, as described above, wherein Xaa2 and Xaa2* are independently selected from Trp, analogs of Trp, other amino acids or aromatic amino acid analogs, and
Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in the present invention. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus Unless otherwise indicated, peptides in Table 1 are amidated at the C-terminus Bold text is used to indicate certain modifications. Activity relative to compstatin is based on published data and assays described therein (WO2004/026328, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 1 are cyclized via a disulfide bond between the two Cys residues when used in the therapeutic compositions and methods of the invention. Alternate means for cyclizing the peptides are also within the scope of the invention.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3 × more |
| Ac-V4Y/H9A | Ac-ICVYQDWGAHRCT-CONH2 | 10 | 14 × xmore |
| Ac-V4W/H9A-OH | Ac-ICVWQDWGAHRCT-COOH | 11 | 27 × xmore |
| Ac-V4W/H9A | Ac-ICVWQDWGAHRCT-CONH2 | 12 | 45 × xmore |
| Ac-V4W/H9A/T13dT-OH | Ac-ICVWQDWGAHRCdT-COOH | 13 | 55 × more |
| Ac-V4(2-Nal)/H9A | Ac-ICV(2-Nal)QDWGAHRCT-CONH2 | 14 | 99 × more |
| Ac V4(2-Nal)/H9A-OH | Ac-ICV(2-Nal)QDWGAHRCT-COOH | 15 | 38 × more |
| Ac V4(1-Nal)/H9A-OH | Ac-ICV(1-Nal)QDWGAHRCT-COOH | 16 | 30 × more |
| Ac-V42Ig1/H9A | Ac-ICV(2-Igl)QDWGAHRCT-CONH2 | 17 | 39 × more |
| Ac-V42Ig1/H9A-OH | Ac-ICV(2-Igl)QDWGAHRCT-COOH | 18 | 37 × more |
| Ac-V4Dht/H9A-OH | Ac-ICVDhtQDWGAHRCT-COOH | 19 | 5 × more |
| Ac-V4(Bpa)/H9A-OH | Ac-ICV(Bpa)QDWGAHRCT-COOH | 20 | 49 × more |
| Ac-V4(Bpa)/H9A | Ac-ICV(Bpa)QDWGAHRCT-CONH2 | 21 | 86 × more |
| Ac-V4(Bta)/H9A-OH | Ac-ICV(Bta)QDWGAHRCT-COOH | 22 | 65 × more |
| Ac-V4(Bta)/H9A | Ac-ICV(Bta)QDWGAHRCT-CONH2 | 23 | 64 × more |
| Ac-V4W/H9(2-Abu) | Ac-ICVWQDWG(2-Abu)HRCT-CONH2 | 24 | 64 × more |
| +G/V4W/H9A +AN-OH | H-GICVWQDWGAHRCTAN-COOH | 25 | 38 × more |
| Ac-V4(5fW)/H9A | Ac-ICV(5fW)QDWGAHRCT-CONH2 | 26 | 31 × more |
| Ac-V4(5-MeW)/H9A | Ac-ICV(5-methyl-W)QDWGAHRCT-CONH2 | 27 | 67 × more |
| Ac-V4(1-MeW)/H9A | Ac-ICV(1-methyl-W)QDWGAHRCT-CONH2 | 28 | 264 × more |
| Ac-V4W/W7(5fW)/H9A | Ac-ICVWQD(5fW)GAHRCT-CONH2 | 29 | 121 × more |
| Ac-V4(5fW)/W7(5fW)/H9A | Ac-ICV(5fW)QD(5fW)GAHRCT-CONH2 | 30 | NA |
| Ac-V4(5-MeW)/W7(5fW)H9A | Ac-ICV(5-methyl-W)QD(5fW)GAHRCT- | 31 | NA |
| | CONH2 | | |
| Ac-V4(1MeW)/W7(5fW)/H9A | Ac-ICV(1-methyl-W)QD(5fW)GAHRCT- | 32 | 264 × more |
| | CONH2 | | |
| +G/V4(6fW)/W7(6fW)H9A+N- | H-GICV(6fW)QD(6fW)GAHRCTN-COOH | 132 | 126 × more |
| OH | | | |
| Ac-V4(1-formyl-W)/H9A | Ac-ICV(1-formyl-W)QDWGAHRCT-CONH2 | 133 | 264 × more |
| Ac-V4(5-methoxy-W)/H9A | Ac-ICV(1-methyoxy-W)QDWGAHRCT- | 134 | 76 × more |
| | CONH2 | | |
| G/V4(5f-W)/W7(5fW)/H9A+N- | H-GICV(5fW)QD(5fW)GAHRCTN-COOH | 135 | 112 × more |
| OH | | | |

NA = not available

In certain embodiments of the compositions and methods of the invention the compstatin analog has a sequence selected from sequences 9-32 or 132-135. In certain embodiments of the compositions and methods of the invention the compstatin analog has a sequence selected from SEQ ID NOs: 14, 21, 28, 29, 32, 132, 133, and 135. In certain embodiments of the compositions and methods of the invention the compstatin analog has a sequence selected from SEQ ID NOs: 30 and 31. In one embodiment of the compositions and methods of the invention the compstatin analog has a sequence of SEQ ID NO: 28. In one embodiment of the methods of the invention the compstatin analog has a sequence of SEQ ID NO: 32. In one embodiment of the methods of the invention the compstatin analog has a sequence of SEQ ID NO: 133. In one embodiment of the methods of the invention the compstatin analog has a sequence of SEQ ID NO: 135.

In certain embodiments of the compositions and methods of the invention the compstatin analog has a sequence as set forth in Table 1, but where the Ac— group is replaced by an alternate blocking moiety B¹, as described above. In some embodiments the —NH₂ group is replaced by an alternate blocking moiety B², as described above.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 1, e.g., SEQ ID NO: 14, 21, 28, 29, 32, 132, 133, or 135 in a peptide-C3 structure, e.g., a crystal structure. In certain embodiments the compstatin analog is a compound that binds to the binding site of a peptide having SEQ ID NO: 30 or 31 in a peptide-C3 structure, e.g., a crystal structure. In certain embodiments the compstatin analog is a compound that could substitute for the peptide of SEQ ID NO: 9-32, e.g., SEQ ID NO: 14, 21, 28, 32, 132, 133, or 135 in a peptide-C3 structure and would form substantially the same intermolecular contacts with C3 as the peptide. In certain embodiments the compstatin analog is a compound that could substitute for the peptide of SEQ ID NO: 30 or 31 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof One of skill in the art would be able to use molecular modeling software programs to predict whether a compound would form substantially the same intermolecular contacts with C3 as would compstatin or a peptide having the sequence of any of the peptides in Table 1, e.g., SEQ ID NO: 14, 21, 28, 29, 32, 132, 133, or 135, or in some embodiments SEQ ID NO: 30 or 31.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, and/or WO2004026328. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

Compstatin Mimetics

The structure of compstatin is known in the art, and NMR structures for a number of compstatin analogs having higher activity than compstatin are also known (Malik, supra). Structural information may be used to design compstatin mimetics.

In one embodiment, the compstatin mimetic is any compound that competes with compstatin or any compstatin analog (e.g., a compstatin analog whose sequence is set forth in Table 1) for binding to C3 or a fragment thereof (such as a 40 kD fragment of the β chain to which compstatin binds). In some embodiments, the compstatin mimetic has an activity equal to or greater than that of compstatin. In some embodiments, the compstatin mimetic is more stable, orally available, or has a better bioavailability than compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In certain embodiments the compstatin mimetic is a compound that binds to the binding site of a peptide having a sequence set forth in Table 1, e.g., SEQ ID NO: 14, 21, 28, 29, 32, 132, 133, or 135, or in certain embodiments SEQ ID NO: 30 or 31, in a peptide-C3 structure. In certain embodiments the compstatin mimetic is a compound that could substitute for a peptide having a sequence set forth in Table 1, e.g., SEQ ID NO: 14, 21, 28, 29, 32, 132, 133, or 135, or in certain embodiments SEQ ID NO: 30 or 31, in a peptide-C3 structure and would form substantially the same intermolecular contacts with C3 as the peptide. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455), Eguchi M, Kahn M., Mini Rev Med Chem., 2(5):447-62, 2002. Of particular relevance to the present invention, the design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Other Compounds that Inhibit C3

Other compounds, e.g., polypeptides, small molecules, monoclonal antibodies, nucleic acids such as aptamers, etc., that bind to C3 or C3a receptors (C3aR) are of use in certain embodiments of the invention. For example, U.S. Pat. No. 5,942,405 discloses C3aR antagonists. Aptamers that bind to and inhibit complement-related proteins may be identified using methods such as SELEX. For example, U.S. Pat. Pub. No. 20030191084 discloses aptamers that bind to Clq, aptamers that bind to C3, and aptamers that bind to C5.

It will be appreciated that chemical modifications of nucleic acids, e.g., aptamers, can be made to increase their in vivo stability or to enhance or mediate their delivery in vivo. See, e.g., U.S. Pat. Nos. 5,660,985; 5,958,691; 5,660,985; U.S. Pat. No. 5,958,691; U.S. Pat. No. 5,698,687; U.S. Pat. No. 5,817,635; U.S. Pat. No. 5,672,695, and PCT Publication WO 1992/07065; WO/2005/113813. Modifications include, but are not limited to, 2′-position sugar modifications, purine or pyrimidine modifications, e.g., 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, etc. Modifications can also include 3′ and/or 5′ modifications such as capping, e.g., 5′ and 3′ phosphorothioate capping and/or 3′-3′ inverted phosphodiester linkage at the 3′ end. 2′-fluoro (2′-F) and/or 2′-amino (2′-NH₂) and/or 2′-O-methyl (2′-OMe) modification of some or all of the nucleotides may be employed. In some embodiments a nucleic acid, e.g., an aptamer, is conjugated to a non-nucleic acid moiety, e.g., polyethylene glycol (PEG), etc.

Compounds that Inhibit Factor B Activation or Activity

In certain embodiments the complement inhibitor inhibits activation of factor B. For example, the complement inhibitor may bind to factor B. Exemplary agents include antibodies, antibody fragments, peptides, small molecules, and aptamers. Exemplary antibodies that inhibit factor B are described in U.S. Pat. Pub. No. 20050260198. In certain embodiments the isolated antibody or antigen-binding fragment selectively binds to factor B within the third short consensus repeat (SCR) domain. In certain embodiments the antibody prevents formation of a C3bBb complex. In certain embodiments the antibody or antigen-binding fragment prevents or inhibits cleavage of factor B by factor D. In certain embodiments the complement inhibitor is an antibody, small molecule, aptamer, or polypeptide that binds to substantially the same binding site on factor B as an antibody described in U.S. Pat. Pub. No. 20050260198. Use of peptides that bind to and inhibit factor B, which may be identified using methods such as phage display, is within the scope of the invention. Use of aptamers that bind to and inhibit factor B, which may be identified using methods such as SELEX, is within the scope of the invention.

Compounds that Inhibit Factor D Activity

In certain embodiments the complement inhibitor inhibits factor D. For example, the complement inhibitor may bind to factor D. Exemplary agents include antibodies, antibody fragments, peptides, small molecules, and aptamers. While factor D has been suggested as a desirable target for systemic complement inhibition as a result of its relatively low serum concentration and ability to inhibit alternative pathway activation, it is believed that the present disclosure is the first to specifically focus attention on the therapeutic potential of locally administered agents that inhibit factor D. Exemplary antibodies that inhibit factor D are described in U.S. Pat. No. 7,112,327. In certain embodiments the complement inhibitor is an antibody, small molecule, aptamer, or polypeptide that binds to substantially the same binding site on factor D as an antibody described in U.S. Pat. No. 7,112,327. Exemplary polypeptides that inhibit alternative pathway activation and are believed to inhibit factor D are disclosed in U.S. Pub. No. 20040038869. Use of peptides that bind to and inhibit factor D, which may be identified using methods such as phage display, is within the scope of the invention. Use of aptamers that bind to and inhibit factor D, which may be identified using methods such as SELEX, is within the scope of the invention.

Compounds that Inhibit C5 Activation or Activity

In certain embodiments the complement inhibitor inhibits activation of C5. For example, the complement inhibitor may bind to C5. Exemplary agents include antibodies, antibody fragments, polypeptides, small molecules, and aptamers. Exemplary antibodies are described in U.S. Pat. No. 6,534,058. Exemplary compounds that bind to and inhibit C5 are described in U.S. Pat. Pub. Nos. 20050090448 and 20060115476. In certain embodiments the complement inhibitor is an antibody, small molecule, aptamer, or polypeptide that binds to substantially the same binding site on C5 as an antibody described in U.S. Pat. No. 6,534,058 or a peptide described in U.S. Ser. No, 10/937,912. U.S. Pat. Pub. No. 20060105980 discloses aptamers that bind to and inhibit C5.

In other embodiments the complement inhibitor is an antagonist of a C5a receptor (C5aR). Exemplary C5a receptor antagonists include a variety of small cyclic peptides such as those described in U.S. Pat. No. 6,821,950; U.S. Ser. No. 11/375,587; and/or PCT/US06/08960 (WO2006/099330), or a peptidomimetic thereof. Sequences of certain peptides that bind to and, in some instances antagonize, mammalian C5aR are presented in Table 2 (wherein [ ] represents cyclization, e.g., via an amide bond, such as between the ornithine side chain and carboxyl-terminal residue in certain of the peptides). See also U.S. Ser. No. 11/375,587 and PCT/US06/08960 (WO2006099330); Huber-Lang, M., et al., FASEB Journal. 2002; 16:1567-1574; March, D., et al., Mol. Pharmacol., 65: 868-879, 2004. In certain embodiments a cyclic peptide comprising the sequence [OPdChaWR] (SEQ ID NO: 130) is used. In certain embodiments a peptide comprising the sequence Xaa[OPdChaWR] (SEQ ID NO: 131) wherein Xaa is a standard or nonstandard aromatic amino acid is used. In certain embodiments a peptide peptide selected from the group consisting of: SEQ ID NOs: 57, 58, 77, and 78 is used.

TABLE 2
Peptides that bind to mammalian C5aR

| SEQ ID NO: | Sequence |
|---|---|
| 33 | (ISHKDMQLGR) |
| 34 | YSFKDMQLGR |
| 35 | YSFKDMPLaR (a = D-ala) |
| 36 | YSFKPMPLaR (a = D-ala) |
| 37 | RAARISLGPRahxYSFKPMPLaR (ahx = Acp; |
| | a = D-ala) |
| 38 | KYKHSVVKKahxYSFKPMPLaR (ahx = Acp; |
| | a = D-ala) |
| 39 | MeFKP(dCha)WR |
| 40 | MeFKP(dCha)WR-CONH₂ |
| 41 | MeFKP(dCha)WR |
| 42 | MeFKPLWR |
| 43 | AcF-[KPdChaWR](R) |
| 44 | AcF-[OPdChaWR](R) |
| 45 | F-[XPdChaWR](S) |
| 46 | F-[XPdChaWR](R) |
| 47 | F-[X²PdChaWR](S) |
| 48 | F-[X²PdChaWR](R) |
| 49 | AcF-[KPdChaWR](S) |
| 50 | AcF-[OPdChaWR](S) |
| 51 | [FWPdChaWR] |
| 52 | AcF-[KMdChaWR] |
| 53 | AcF-[KKdChaWR] |
| 54 | AcF-[XPdChaWR] |
| 55 | AcF-[X²PdChaWR] |
| 56 | AcKF-[OPdChaWR] |
| 57 | F-[OPdChaWR] |
| 58 | F-[KPdChaWR] |
| 59 | F-[OPdChaWR] |
| 60 | F-[KPdChaWR] |
| 61 | AcF-[OPdChaWR] |
| 62 | Mes-F-[OPdChaWR] |
| 63 | Tos-F-[OPdChaWR] |
| 64 | Suc-F-[OPdChaWR] |
| 65 | MeSuc-F-[OPdChaWR] |
| 66 | Ahx-F-[OPdChaWR] |
| 67 | Bn-F-[OPdChaWR] |
| 68 | Iso-F-[OPdChaWR] |
| 69 | Ac-G-[OPdChaWR] |
| 70 | Ac-f-[OPdChaWR] |
| 71 | Ac-Phg-[OPdChaWR] |
| 72 | Ac-hPhe-[OPdChaWR] |
| 73 | Ac-(o-fl)F-[OPdChaWR] |
| 74 | Ac-(m-fl)F-[OPdChaWR] |
| 75 | Ac-Y-[OPdChaWR] |
| 76 | H-[OPdChaWR] |
| 77 | Cin-[OPdChaWR] |
| 78 | HCin-[OPdChaWR] |
| 79 | AcF-[OVdChaWR] |
| 80 | AcF-[OFdChaWR] |
| 81 | AcF-[OIdChaWR] |
| 82 | AcF-[OHypdChaWR] |
| 83 | AcF-[OThpdChaWR] |
| 84 | AcF-[(δ-N-Me)OPdChaWR] |
| 85 | AcF-[OPGWR] |
| 86 | AcF-[OPdAlaWR] |
| 87 | AcF-[OPdValWR] |
| 88 | AcF-[OPdLeuWR] |
| 89 | AcF-[OPdNleWR] |
| 90 | AcF-[OPdPheWR] |
| 91 | AcF-[OP(N-Me)fWR] |
| 92 | AcF-[OPdhChaWR] |
| 93 | AcF-[OPdTyrWR] |
| 94 | AcF-[OPdArgWR] |
| 95 | AcF-[OPdTrpWR] |
| 96 | AcF-[OPdTicWR] |
| 97 | AcF-[OPdAicWR] |
| 98 | AcF-[OPdChaLR] |
| 99 | AcF-[OPdChaChaR] |
| 100 | AcF-[OPdChaHR] |
| 101 | AcF-[OPdChaFR] |
| 102 | AcF-[OPdChahFR] |
| 103 | AcF-[OPdCha(N-Me)FR] |
| 104 | AcF-[OPdChawR] |
| 105 | AcF-[OPdChaBtaR] |
| 106 | AcF-[OPdCha1Na1R] |
| 107 | AcF-[OPdCha2Na1R] |
| 108 | AcF-[OPdChaTicR] |
| 109 | AcF-[OPdChaFlaR] |
| 110 | AcF-[OPdChaWCit] |
| 111 | AcF-[OPdChaWhArg] |
| 112 | AcF-[OPdChaWK] |
| 113 | AcF-[OPfFR] |
| 114 | AcF-[OPf1Na1R] |
| 115 | AcF-[OPfYR] |
| 116 | AcF-[OPdChaF(pC1)F] |
| 117 | AcF-[OPdChaF(pNH₂)F] |
| 118 | AcF-[OPdChaFhF] |
| 119 | AcF-[OPdArgFR] |
| 120 | AcF-[OPGmbTyrR] |
| 121 | Ac-F-[OPdChaWR] |
| 122 | Ac-F-[OPdChaanthrylAR] |
| 123 | Ac-F-[OPdanthrylAWR] |
| 124 | Ac-F-[OanthrylAdChaWR] |
| 125 | Ac-anthrylA[OPdChaWR] |
| 126 | Fmoc-anthrylA-[OPdChaWR] |
| 127 | Fmoc-F-[OPdChaWR] |
| 128 | Fmoc-[OPdChaWR] |
| 129 | NH₂-[OPdChaWR] |

### Abbreviations

X═(CH₂)—NH₂; X²═(CH₂)₂—NH₂; Mes, mesyl; Tos, tosyl; Sue, succinyl; MeSue, methyl succinyl; Ahx, aminohexanoyl; Bn, benzoyl; Iso, isovaleroyl; Phg, phenylglycine; hPhe or hF, homophenylalanine; (o-fl)F, ortho-fluoro phenylalanine; (m-fl)F, meta-fluoro phenylalanine; Cha, cyclohexylalanine; Cin, cinnamoyl; Hein, hydrocinnamoyl; Hyp, 4-trans-hydroxyproline; Thp, 4-cis-thioproline; (δ-N-Me)O, δ-N-methylated ornithine; (N-Me)f, N-methyl-D-phenylalanine; hCha, homocyclohexylalanine; Tic, tetrahydroisoquinolone; Aic, aminoindanecarboxylic acid; hPhe, homophenylalanine; (N-Me)F, N-methyl-phenylalanine; Bta, benzothiazolealanine; 1Nal, 1-Naphthylalanine; 2-Nal, 2-Naphthylalanine; Fla, fluorophenylalanine; Cit; citrulline; hArg, homoarginine; (pC1)F, para-chlorophenylalanine; (pNH₂)F, para-aminophenylalanine; mbTyr, metabenzyltryosine; anthrylA, anthrylalanine; 9-Fluorenylmethyloxycarbonyl.

In certain embodiments of the invention the complement inhibitor does not bind to C5, C5a, or C5aR. In certain embodiments of the invention the complement inhibitor does not inhibit activation of C5.

Multimodal Complement Inhibitors

In certain embodiments of the invention the complement inhibitor binds to more than one complement protein and/or inhibits more than one step in a complement activation pathway. Such complement inhibitors are referred to herein as “multimodal”. One aspect of this invention is the recognition of the advantages of inhibiting complement activation at multiple points in the complement activation pathway in a trauma patient by administration of a multimodal complement inhibitor. In certain embodiments of the invention the complement inhibitor is a virus complement control protein (VCCP). The invention specifically contemplates use of any of the agents described in U.S. Ser. No. 11/247,886 and PCT/US2005/36547, filed Oct. 8, 2005. Poxviruses and herpesviruses are families of large, complex viruses with a linear double-stranded DNA genome. Certain of these viruses encode immunomodulatory proteins that are believed to play a role in pathogenesis by subverting one or more aspects of the normal immune response and/or fostering development of a more favorable environment in the host organism (Kotwal, G J, Immunology Today, 21(5), 242-248, 2000). Among these are VCCPs. Poxvirus complement control proteins are members of the complement control protein (CCP) superfamily and typically contain 4 SCR modules. These proteins have features that make them advantageous for complement inhibition in accordance with the present invention. In certain embodiments the VCCP is a poxvirus complement control protein (PVCCP). The PVCCP can comprise a sequence encoded by, e.g., vaccinia virus, variola major virus, variola minor virus, cowpox virus, monkeypox virus, ectromelia virus, rabbitpox virus, myxoma virus, Yaba-like disease virus, or swinepox virus. In other embodiments the VCCP is a herpesvirus complement control protein (HVCCP). The HVCCP can comprise a sequence encoded by a Macaca fuscata rhadinovirus, cercopithecine herpesvirus 17, or human herpes virus 8. In other embodiments the HVCCP comprises a sequence encoded by herpes simplex virus saimiri ORF 4 or ORF 15 (Albrecht, J C. & Fleckenstein, B., J. Virol., 66, 3937-3940, 1992; Albrecht, J., et al., Virology, 190, 527-530, 1992).

The VCCP may inhibit the classical complement pathway, the alternate complement pathway, the lectin pathway, or any two or more of these. In certain embodiments of the invention the VCCP, e.g., a PVCCP, binds to C3b, C4b, or both. In certain embodiments of the invention the PVCCP comprises one or more putative heparin binding sites (K/R-X-K/R) and/or possesses an overall positive charge. In some embodiments the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or can be a mature form (i.e., lacking the signal sequence).

VCP is described in U.S. Pat. Nos. 5,157,110 and 6,140,472, and in Kotwal, G K, et al., Nature, 355, 176-178, 1988. FIGS. 3A and 3B of U.S. Ser. No. 11/247,886 and PCT/US2005/36547 (WO2006042252) show the sequence of the precursor and mature VCP proteins, respectively. VCP blocks complement activation at multiple steps and reduces levels of the proinflammatory chemotactic factors C3a, C4a, and C5a.

Variola virus major and minor encode proteins that are highly homologous to VCP and are referred to as smallpox inhibitor of complement enzymes (SPICE) (Rosengard, AM, et al., Proc. Natl. Acad. Sci., 99(13), 8803-8813. U.S. Pat. No. 6,551,595). SPICE from various variola strains sequenced to date differs from VCP by about 5% (e.g., about 11 amino acid differences). Similarly to VCP, SPICE binds to C3b and C4b and causes their degradation, acting as a cofactor for factor I. However, SPICE degrades C3b approximately 100 times as fast as VCP and degrades C4b approximately 6 times as fast as VCP. The amino acid sequence of SPICE is presented in FIG. 6 (SEQ ID NO: 12) of U.S. Ser. No. 11/247,886 and PCT/US2005/36547 (WO2006042252)

It will be appreciated that the exact sequence of complement control proteins identified in different virus isolates may differ slightly. Use of such proteins falsl within the scope of the present invention. Complement control proteins from any such isolate may be used, provided that the protein has not undergone a mutation that substantially abolishes its activity. Thus the sequence of a VCCP such as SPICE or VCP may differ from the exact sequences presented herein or under the accession numbers listed in Table 2. It will also be appreciated that a number of amino acid alterations, e.g., additions, deletions, or substitutions such as conservative amino acid substitutions, may be made in a typical polypeptide such as a VCCP without significantly affecting its activity, such that the resulting protein is considered equivalent to the original polypeptide. In some embodiments the VCCP is at least 90%, 95%, 98%, or 99% identical in sequence to a sequence identified by accession number in Table 3.

TABLE 3
Representative Viral Complement Control Proteins

| Virus | Protein | Accession | Virus Type |
|---|---|---|---|
| Variola | D12L | NP_042056 | Orthopoxvirus |
| | D15L (SPICE) | AAA69423 | Orthopoxvirus |
| Vaccinia | VCP | AAO89304 | Orthopoxvirus |
| Cowpox | CPXV034 | AAM13481 | Orthopoxvirus |
| | C17L | CAA64102 | Orthopoxvirus |
| Monkeypox | D14L | AAV84857 | Orthopoxvirus |
| Ectromelia virus | Complement | CAE00484 | Orthopoxvirus |
| | control protein | | |
| Rabbitpox | RPXV017 | AAS49730 | Orthopoxvirus |
| Macaca fuscata | JM4 | AAS99981 | Rhadinavirus |
| rhadinovirus | | | (Herpesvirus) |
| Cercopithecine | Complement binding | NP_570746 | Herpesvirus |
| herpesvirus 17 | protein (ORF4) | | |
| Human herpes | Complement binding | AAB62602 | Herpesvirus |
| virus 8 | protein (ORF4) | | |

Additional Complement Inhibiting Agents, Combinations, and Modifications

A variety of other complement inhibitors known in the art can be used in various embodiments of the invention. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 130) or KTISKAKGQPREPQVYT (SEQ ID NO: 131) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

In some embodiments the complement inhibitor is a mammalian, e.g., human, complement regulatory protein such as CFH, CFI, CR1, DAF, CD59, C4bp, or complement receptor 2 inhibitor trispanning (CRIT; Inal, J., et al, J Immunol., 174(1):356-66, 2005). In some embodiments of the invention the complement inhibitor is a fragment or variant of any of the foregoing polypeptides that retains at least 20%, e.g., at least 50%, of the complement inhibitory activity of the naturally occurring polypeptide. In some embodiments the fragment comprises at least 30%, 40%, 50%, 60%, 70%, 80%, 90%, or 95% of the full length polypeptide. In some embodiments the variant is at least 30%, 40%, 50%, 60%, 70%, 80%, 90%, or 95% identical to a naturally occurring mammalian, e.g., human, complement regulatory protein. In some embodiments the variant comprises a fragment at least 50%, 60%, 70%, 80%, 90%, or 95% as long as a mammalian, e.g., human complement regulatory protein, wherein the fragment is at least 30%, 40%, 50%, 60%, 70%, 80%, 90%, or 95% identical to the naturally occurring mammalian, e.g., human, complement regulatory protein. In some embodiments, the fragment or variant has a higher complement inhibiting activity on a weight basis and/or a molar basis than the naturally occurring polypeptide. In some embodiments the complement inhibitor is a polypeptide comprising at least 1, 2, 3, or 4 SCRs. For example, the polypeptide may comprise between 4 and 100 SCRs.

Polypeptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides. Polypeptides may contain one or more non-natually occurring amino acids or amino acid analogs or be otherwise modified. Activity of certain polypeptides is at least partly dependent on their glycosylation state. It may be desirable to produce such polypeptides in systems that provide for glycosylation similar or substantially identical to that found in mammals, e.g., humans. For example, mammalian expression systems or modified lower eukaryotic expression systems (e.g., fungal expression systems), that provide for mammalian-like glycosylation can be used. See, e.g., U.S. Pub. Nos. 20060177898 and 20070184063.

In some embodiments of the invention the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCR1), for example, are of use in the invention. For example the compounds known as TP10 or TP20 (Avant Therapeutics) can be used. C1 inhibitor (C1-INH) is also of use. In some embodiments a soluble complement control protein, e.g., CFH, is used. In some embodiments of the invention the polypeptide is modified to increase its solubility.

Combination therapy using two or more complement inhibitors is within the scope of the invention. The two or more complement inhibitors may be provided in the same composition or separately. In one embodiment at least two of the complement inhibitors are peptides, each having a length between 5 and 50 amino acids, optionally cyclic. In certain embodiments the complement inhibitors bind to two or more different complement components. In certain embodiments the complement inhibitors bind to two or more different soluble complement proteins. In certain embodiments the complement inhibitors inhibit activation or activity of at least two complement proteins selected from C3, C5, factor B, and factor D. In certain embodiments a first complement inhibitor inhibits activation or activity of C3 and a second complement inhibitor inhibit activation or activity of a complement protein selected from C5, factor B, and factor D. In certain embodiments a first complement inhibitor inhibits activation or activity of C3 and a second complement inhibitor inhibit activation or activity of a complement protein selected from factor B and factor D. In certain embodiments a first complement inhibitor inhibits activation or activity of C3 and a second complement inhibitor inhibit activation or activity of a complement protein selected from C5 and C5a. For example, in certain embodiments a first complement inhibitor inhibits activation or activity of C3 and a second complement inhibitor binds to and inhibits C5aR. In certain embodiments of the invention a first complement inhibitor binds to C3 and a second complement inhibitor is a complement regulatory protein that contributes to dissociation, inactivation or degradation of a convertase or activated complement component. For example, the complement regulatory protein may be soluble CR1, DAF, MCP, CFH, or CFI. In some embodiments the complement inhibitor is a chimeric protein comprising at least a portion of a first complement regulatory protein (e.g., DAF) and at least a portion of a second complement regulatory protein (e.g., MCP), such as the complement inhibitor known as CAB-2, or a variant thereof (see, e.g., U.S. Pat. No. 5,679,546. The variant may have, e.g., at least 25% of the complement inhibiting ability of CAB-2.

Complement inhibitors, optionally linked to a binding moiety, can be modified by addition of a molecule such as polyethylene glycol (PEG) or similar molecules to stabilize the compound, reduce its immunogenicity, increase or decrease its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002; Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures.

In certain embodiments the complement inhibitor is a multivalent compound comprising a plurality of complement inhibitor moieties covalently or noncovalently linked to a polymeric backbone or scaffold. The complement inhibitor moieties may be the same or different. A complement inhibitor may comprise or be modified to comprise a reactive functional group or be attached to a linker comprising a reactive functional group. The reactive functional group facilitates the attachment of the complement inhibitor to the polymeric backbone. The complement inhibitor can be any of the complement inhibitors described herein. It will be appreciated that following attachment to the polymeric backbone, the structure of the complement inhibitor moiety will differ slightly from that of the complement inhibitors described herein. For example, a complement inhibitor comprising an amine (NH₂) group, represented as NH₂—R¹, may react with a moiety comprising a carboxylic acid (COOH), represented as R²—(C══O)OH to form a conjugate having formula R²—(C═O)—NH—R¹, in which one of the hydrogens present in the complement inhibitor is no longer present and a new covalent bond (C—N) has been formed. Thus the term “complement inhibitor moiety” includes molecules having the precise formula of a complement inhibitor as described herein as well as molecular structures in which a functional group of a complement inhibitor has reacted with a second functional group, which typically entails loss of at least one atom or group of atoms that was present in the complement inhibitor molecule prior to the reaction and formation of a new covalent bond. The new covalent bond is formed between an atom that was previously attached to one of the atoms that is lost from the complement inhibitor and an atom to which the complement inhibitor becomes attached.

The complement inhibitor moieties can be identical or different. In certain embodiments of the invention the multivalent compound comprises multiple instances, or copies, of a single complement inhibitor moiety. In other embodiments of the invention the multivalent compound comprises one or more instances of each of two of more non-identical complement inhibitor moieties, e.g., 3, 4, 5, or more different complement inhibitor moieties. In certain embodiments of the invention the number of complement inhibitor moieties (“n”) is between 2 and 6. In other embodiments of the invention n is between 7 and 20. In other embodiments of the invention n is between 20 and 100. In other embodiments n is between 100 and 1,000. In other embodiments of the invention n is between 1,000 and 10,000. In other embodiments n is between 10,000 and 50,000. In other embodiments n is between 50,000 and 100,000. In other embodiments n is between 100,000 and 1,000,000.

The complement inhibitor moieties may be attached directly to the polymeric scaffold or may be attached via a linking moiety that connects the complement inhibitor moiety to the polymeric scaffold. The linking moiety may be attached to a single complement inhibitor moiety and to the polymeric scaffold. Alternately, a linking moiety may have multiple complement inhibitor moieties joined thereto so that the linking moiety attaches multiple compstatin analog moieties to the polymeric scaffold.

In one embodiment, the complement inhibitor comprises an amino acid having a side chain comprising a primary or secondary amine, e.g., a Lys residue. For example, a Lys residue, or a sequence comprising a Lys residue, is added at the C-terminus of the complement inhibitor. In one embodiment, the Lys residue is separated from the domain responsible for the biological activity of the complement inhibitor by a rigid or flexible spacer. For example, the Lys residue may be separated from a cyclic portion of the molecule by a spacer. The spacer may, for example, be a substituted or unsubstitued, saturated or unsaturated alkyl chain. The length of the alkyl chain may be, e.g., between 2 and 20 carbon atoms.

In certain embodiments of the invention the linker moiety or spacer comprises or consists of a peptide. The peptide may be, e.g., between 1 and 20 amino acids in length, e.g., between 4 and 20 amino acids in length. Suitable peptides can comprise or consist of multiple Gly residues, Ser residues, or both. In certain embodiments the peptide comprises or consists of between 1 and 15 amino acids, e.g., between 2 and 8 amino acids. In certain embodiments the peptide has the formula Xₙ where each X is independently glycine or serine, and n is between 1 and 15. Exemplary peptides include (G)ₙ, wherein n=4, 5, or 6. Additional exemplary peptides include any of the foregoing, wherein any 1, 2, 3, or 4 of the glycines are replaced by serine. It will be appreciated that the terminal residues of the peptides may or may not participate in peptide bonds

Any of a variety of polymeric backbones or scaffolds could be used. For example, the polymeric backbone or scaffold may be a polyamide, polysaccharide, polyanhydride, polyacrylamide, polymethacrylated, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the complement inhibitors. In one embodiment, the complement inhibitor comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the complement inhibitors and the resulting monomers are polymerized. In another embodiment, short chains are prepolymerized, functionalized, and then a mixture of short chains of different composition are assembled into longer polymers.

Bifunctional Complement Inhibitors

Bifunctional complement inhibitors are an aspect of the invention. In certain embodiments the complement inhibitor comprises a first complement inhibitor moiety that inhibits a first complement component and a second complement inhibitor moiety that inhibits a receptor for a second complement component. In some embodiments of the invention the first moiety inhibits C3 and the second moiety inhibits the C5a receptor. The first and second moieties may be any of the complement inhibitor moieties described herein. In some embodiments, the complement inhibitor moieties are cyclic peptides. In some embodiments one moiety, e.g., the 5′ moiety, comprises a compstatin analog and the second moiety, e.g., the 3′ moiety, comprises a C5a receptor antagonist. In some embodiments, the first moiety comprises a peptide selected from 14, 21, 28, 29, 32, 132, 133, and 135, and the second moiety comprises a peptide selected from SEQ ID NOs: 57, 58, 77, and 78.

In some embodiments the first moiety comprises or consists of SEQ ID NO: 28 and the second moiety comprises or consists of SEQ ID NO: 57. In some embodiments the first moiety comprises or consists of SEQ ID NO: 32 and the second moiety comprises or consists of SEQ ID NO: 57. In some embodiments the first moiety comprises or consists of SEQ ID NO: 133 and the second moiety comprises or consists of SEQ ID NO: 57.

In some embodiments the first moiety comprises or consists of SEQ ID NO: 28 and the second moiety comprises or consists of SEQ ID NO: 58. In some embodiments the first moiety comprises or consists of SEQ ID NO: 32 and the second moiety comprises or consists of SEQ ID NO: 58. In some embodiments the first moiety comprises or consists of SEQ ID NO: 133 and the second moiety comprises or consists of SEQ ID NO: 58.

In some embodiments the first moiety comprises or consists of SEQ ID NO: 28 and the second moiety comprises or consists of SEQ ID NO: 77. In some embodiments the first moiety comprises or consists of SEQ ID NO: 32 and the second moiety comprises or consists of SEQ ID NO: 77. In some embodiments the first moiety comprises or consists of SEQ ID NO: 133 and the second moiety comprises or consists of SEQ ID NO: 77.

In some embodiments the first moiety comprises or consists of SEQ ID NO: 28 and the second moiety comprises or consists of SEQ ID NO: 78. In some embodiments the first moiety comprises or consists of SEQ ID NO: 32 and the second moiety comprises or consists of SEQ ID NO: 78. In some embodiments the first moiety comprises or consists of SEQ ID NO: 133 and the second moiety comprises or consists of SEQ ID NO: 78.

Optionally in any of the embodiments, the moieties are linked by a linker moiety, In some embodiments the linker moiety comprises or consists of a peptide, e.g., (G)₄, (G)₅, A(G)₄, A(G)₅, A(G)₄A, A(G)₅A, S(G)₄, S(G)₅, etc. In some embodiments the linker moiety contains fewer than 5 amino acids. In some embodiments the linker moiety contains fewer than 10 amino acids. In some embodiments the linker moiety contains fewer between 5 and 10 amino acids. In some embodiments the linker moiety contains more than than 10 amino acids, e.g., between 10 and 20 amino acids. In some embodiments the linker moiety is a non-peptide linker moiety.

In some embodiments of the invention a blocking moiety, e.g., any of the blocking moieties described above, is present at one or both ends of a peptide, while in other embodiments a blocking moiety is not present.

In exemplary embodiments, the bifunctional complement inhibitor comprises or consists of one of the following sequences, where * represents amino acids linked by a disulfide bond and [ ] denotes cyclization and abbreviations are as described above:

(SEQ ID NO: 136)
Ac-IC*V(1-methyl-1-tryptophan)QDWGAHRC*TAGGGGF
[OPdChaWR]
(SEQ ID NO: 137)
Ac-IC*V(1-methyl-1-tryptophan)QDWGAHRC*TAGGGGF
[KPdChaWR]
(SEQ ID NO: 138)
Ac-IC*V(1-methy1-1-tryptophan)QDWG5fWHRC*TAGGGGF
[OPdChaWR]
(SEQ ID NO: 139)
Ac-IC*V(1-methy1-1-tryptophan)QDWG5fWHRC*TAGGGGF
[KPdChaWR]
(SEQ ID NO: 140)
Ac-IC*V(1-methyl-1-tryptophan)QDWGAHRC*TAGGGGF
[OPdChaWR]
(SEQ ID NO: 141)
Ac-IC*V(1-methyl-1-tryptophan)QDWGAHRC*TAGGGGF
[KPdChaWR]
(SEQ ID NO: 142)
Ac-IC*V(1-methyl-1-tryptophan)QDWG5fWHRC*TAGGGGF
[OPdChaWR]
(SEQ ID NO: 143)
Ac-IC*V(1-methyl-1-tryptophan)QDWG5fWHRC*TAGGGGF
[KPdChaWR]
(SEQ ID NO: 144)
Ac-IC*V(1-formyl-1-tryptophan)QDWGAHRC*TAGGGGF
[OPdChaWR]
(SEQ ID NO: 145)
Ac-IC*V(1-formyl-1-tryptophan)QDWGAHRC*TAGGGGF
[KPdChaWR]
(SEQ ID NO: 146)
Ac-IC*V(1-methyl-1-tryptophan)QDWGAHRC*TAGGGGAF
[OPdChaWR]
(SEQ ID NO: 147)
Ac-IC*V(1-methyl-1-tryptophan)QDWGAHRC*TAGGGGAF
[KPdChaWR]
(SEQ ID NO: 148)
Ac-IC*V(1-methy1-1-tryptophan)QDWG5fWHRC*TAGGGGAF
[OPdChaWR]
(SEQ ID NO: 149)
Ac-IC*V(1-methy1-1-tryptophan)QDWG5fWHRC*TAGGGGAF
[KPdChaWR]
(SEQ ID NO: 150)
Ac-IC*V(1-methyl-1-tryptophan)QDWGAHRC*TAGGGGAF
[OPdChaWR]
(SEQ ID NO: 151)
Ac-IC*V(1-methyl-1-tryptophan)QDWGAHRC*TAGGGGAF
[KPdChaWR]
(SEQ ID NO: 152)
Ac-IC*V(1-methyl-1-tryptophan)QDWG5fWHRC*TAGGGGAF
[OPdChaWR]
(SEQ ID NO: 153)
Ac-IC*V(1-methyl-1-tryptophan)QDWG5fWHRC*TAGGGGAF
[KPdChaWR]
(SEQ ID NO: 154)
Ac-IC*V(1-formyl-1-tryptophan)QDWGAHRC*TAGGGGAF
[OPdChaWR]
(SEQ ID NO: 155)
Ac-IC*V(1-formyl-1-tryptophan)QDWGAHRC*TAGGGGF
[KPdChaWR]

In some embodiments, bifunctional complement inhibitors comprising two cyclic peptides are made by synthesizing the individual peptides, cyclizing them, and then linking them together, thereby forming a peptide comprising two cyclic portions. In some embodiments, bifunctional complement inhibitors comprising two cyclic peptides are made by synthesizing a linear peptide and then cyclizing the two portions, thereby forming a peptide comprising two cyclic portions. The linker moiety, if present, could be part of either peptide, or the two cyclic peptides could be attached to different residues of a linking moiety, e.g., at opposite ends of a linking moiety. Covalent linkages could be made at the N- or C-termini or at side chains of amino acid residues in various embodiments of the invention. The desired compound at any stage of the synthesis procedure may be separated from starting materials, intermediates, and/or undesired products of a synthesis reaction using any suitable method. In some embodiments, separation is performed using chromatography (e.g., high performance liquid chromatography). The desired compound could be purified to a desired degree of purity. For example, the compound could be purified such that the desired compound constitutes at least 50%, 60%, 70%, 80%, 90%, 95%, 96%, 97%, 98%, 99%, or more (by dry weight) of the peptide material in a purified preparation.

Such bifunctional complement inhibitors are of use to treat any complement-mediated disorder. For purposes of convenience, and without limitations, such disorders and conditions may be classified as follows, (i) eye disorders characterized by macular degeneration, choroidal neovascularization, retinal neovascularization, and/or ocular inflammation, such as age-related macular degeneration (AMD), diabetic retinopathy, glaucoma, uveitis, and keratitis; (ii) disorders of the respiratory system such as asthma, chronic obstructive pulmonary disease (COPD), allergic rhinitis, and infection-associated inflammation; (iii) disorders affecting the nervous system such as spinal cord injury, multiple sclerosis, Alzheimer's disease, Parkinson's Disease, stroke, and chronic pain; (iv) inflammatory arthritis such as rheumatoid arthritis, psoriatic arthritis, Reiter's syndrome, juvenile arthritis, and gout; (v) skin disorders such as psoriasis, pemphigus, scleroderma, and lupus; (vi) surgical or non-surgical trauma or complication(s) thereof; (vii) hereditary or acquired angioedema; and (viii) drug hypersensitivity reactions, also known as “pseudoallergy” and other situations leading to undesirable complement activation, e.g., reactions to contrast media, drug formulations containing lipid or polymer vehicles, exposure to implants or indwelling devices having surfaces that activate complement, transplantation, hemodialysis, etc. It will also be understood that the agent may be administered locally or systemically in various embodiments of the invention, and appropriate formulations will be used for such administration. In some embodiments intravenous administration is used. In other embodiments, an agent is administered to an organ, organ system, or site within the body, wherein the organ, organ system, or site within the body is affected by a disorder. For example, agents may be administered by inhalation for disorders affecting the respiratory system. Agents may be injected into a joint space for treatment of arthritis. Agents may be applied topically, e.g., in the form of a cream, lotion, or ointment, for treatment of skin conditions. Agents may be administered intracranially or intrathecally for treatment of disorders affecting the nervous system.

In other embodiments, the bifunctional complement inhibitor is used ex vivo, i.e., outside the body. For example, the bifunctional complement inhibitor may be used to inhibit complement activation in blood products (e.g., whole blood or a fraction thereof such as serum or plasma, red blood cells, leukocytes, or platelets), to inhibit complement activation in cells, tissues, or organs that are to be used for therapy (e.g., for cell therapy (e.g., bone marrow transplant), tissue or organ transplant, etc.). In some embodiments the bifunctional complement inhibitor is used to inhibit complement activation in the setting of hemodialysis.

The invention thus provides an ex vivo composition comprising (a) a bifunctional complement inhibitor of the invention; and (ii) a cell, blood product, tissue, or organ. The composition can further comprise, e.g., a medium suitable for maintaining said cell, blood product, tissue, or organ ex vivo, e.g., prior to administration to a subject. The composition can further comprise, e.g., a compound useful for maintaining said cell, blood product, tissue, or organ ex vivo, e.g., prior to administration to a subject. For example, the composition can comprise a preservative, a buffer, a nutrient, oxygen, a substance that kills, inactivates, or inhibits growth of a pathogen such as a virus, bacterium, or parasite, etc. The organ may be, e.g., any organ that is of use in transplant therapy, e.g., heart, lung, liver, kidney, skin, pancreas or portions thereof such as islets, etc.

The invention also provides a method of inhibiting complement activation in an ex vivo composition comprising a cell, blood product, tissue, or organ, the method comprising maintaing the cell, blood product, tissue, or organ, in a composition comprising a bifunctional complement inhibitor of the invention. In some embodiments the organ or tissue is cryopreserved. In some embodiments the organ is maintained in a functioning state, e.g., perfused with a solution comprising oxygen, blood, and the bifunctional complement inhibitor. In some embodiments the tissue comprises skin, bone, tendon, muscle, nerve, and/or blood vessel.

The invention further provides a method of treating a subject in need thereof using the afore-mentioned ex vivo composition, the method comprising administering the composition to the subject. In some embodiments, the cell or tissue is autologous to the subject, while in other embodiments the cell or tissue is not autologous to the subject.

Targeting Complement Inhibitors to Sites of Injury

The invention provides a composition comprising (i) a complement inhibitor; and (ii) a binding moiety that binds to a biomolecule present at increased levels in injured tissue. In some embodiments, the binding moiety binds to a biomolecule present at increased levels at sites of complement activation. For example, the binding moiety may bind to an activated complement protein or fragment thereof In certain embodiments of the invention the binding moiety and the complement inhibitor are linked. The linkage can be covalent or noncovalent and can be direct or indirect. The binding moiety can be, for example, an antibody or ligand. According to certain embodiments of the invention the component is a cellular marker. The cellular marker can be any marker that is expressed on or at the surface of a cell of an individual who has suffered a traumatic injury at higher levels than would be found in the individual in the absence of said injury. In some embodiments the marker is a neoantigen present on or at the surface of cells that have been subjected to ischemia.

In general, the component can be any molecule present on or at the surface of a cell or noncellular molecular entity or localized extracellularly at a site of injury. By “on or at the surface of the cell or noncellular molecular entity” is meant that the component is accessible to molecules present in the extracellular environment so that it can be recognized and bound by the moiety. The component may be entirely extracellular. The component may be inserted into the cell membrane. In certain embodiments of the invention the component may be partly or entirely within the membrane. As long as a sufficient portion of the component is exposed or accessible so that it can be recognized and bound, it will be said to be present on or at the surface. In certain embodiments of the invention the component is a cellular marker, e.g., a cell type specific marker. Where the target is a molecular entity other than a cell, the component can be any chemical entity present on or at the surface of the molecule that is recognizable by an antibody or ligand.

In certain embodiments of the invention the binding moiety is linked to a compstatin analog. In other embodiments the binding moiety comprises a portion that binds to another molecule to which compstatin or an analog thereof is attached. Suitable binding moieties include antibodies that specifically bind to a cellular marker or noncellular molecular entity present at a site of tissue damage. The linkage between the binding moiety and the compstatin analog can be covalent or noncovalent and can be direct or indirect in various embodiments of the invention.

In embodiments of the invention in which the binding moiety is an antibody, the antibody may be, in various embodiments of the invention, any immunoglobulin or a derivative thereof, which maintains binding ability, or any protein having a binding domain which is homologous or largely homologous to an immunoglobulin binding domain. Such proteins may be derived from natural sources, or partly or wholly synthetically produced (e.g., using recombinant DNA techniques, chemical synthesis, etc.). The antibody can be of any species, e.g., human, rodent, rabbit, goat, chicken, etc. The antibody may be a member of any immunoglobulin class, including any of the human classes: IgG, IgM, IgA, IgD, and IgE. In various embodiments of the invention the antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Monovalent, bivalent or multivalent antibodies can be used. The antibody may be a chimeric or “humanized” antibody in which, for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Preferably the antibody specifically binds to its target on the cell surface, e.g., to a cell-type specific marker. Methods for producing antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture.

In certain embodiments of the invention it is preferable to use F(ab′)2 or F(ab′) fragments rather than antibodies that contain an Fc portion. However, in certain embodiments of the invention it is preferred to use antibodies comprising an Fc domain. F(ab′)₂ fragments can be generated, for example, through the use of an Immunopure F(ab′)₂ Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(a′)₂. The yield of F(a′)₂ resulting from the digestion can be monitored by standard protein gel electrophoresis. F(a′) can be obtained by papain digestion of antibodies, or by reducing the S—S bond in the F(a′)₂.

In various embodiments of the invention an appropriate binding moiety to which a complement inhibitor (e.g., a compstatin analog) is linked can be any molecule that specifically binds to a target molecule (e.g., polypeptide or a portion thereof such as a carbohydrate moiety), through a mechanism other than an antigen-antibody interaction. Such a binding moiety is referred to as a “ligand”. For example, in various embodiments of the invention a ligand can be a polypeptide, peptide, nucleic acid (e.g., DNA or RNA), carbohydrate, lipid or phospholipid, or small molecule (e.g., an organic compound, whether naturally-occurring or artificially created that has relatively low molecular weight and is not a protein, polypeptide, nucleic acid, or lipid, typically with a molecular weight of less than about 1500 g/mol and typically having multiple carbon-carbon bonds).

Ligands may be naturally occurring or synthesized, including molecules whose structure has been invented by man. Examples of ligands include, but are not limited to, hormones, growth factors, neurotransmitters that bind to particular receptors, molecules such as complement receptors, or fragments thereof, that bind to activated complement components or fragments such as iC3b/C3dg. For example, CR2 or a fragment thereof can be used to target a complement inhibitor to a site of complement activation. Bifunctional complement inhibitors comprising a C5aR inhibitor will target cells that express C5aR, e.g., monocytes, leukocytes, etc.

It will be appreciated that fragments or variants of a polypeptide ligand differing in sequence from their naturally occurring counterparts but retaining significant ability to bind to the marker can also be used. It will also be appreciated that fragments or variants of the polypeptide complement inhibitors mentioned above can be used, provided such fragment or variant retains significant activity as a complement inhibitor. For example, a fragment or variant may retain at least 10%, 25%, 50%, or 75% of the binding ability or activity of the polypeptide of which it is a fragment or variant (the “original polypeptide”). In certain embodiments of the invention, a polypeptide variant contains 5 or fewer amino acid differences, 10 or fewer amino acid differences, 25 or fewer amino acid differences, 50 or fewer amino acid differences, or 100 or fewer amino acid differences with respect to the original polyeptpide. In certain embodiments of the invention the number of amino acid differences between a polypeptide ligand or polypeptide complement inhibitor and a fragment or variant thereof for use in the invention is 5% or less, 10% or less, or 25% or less of the total number of amino acids in the original polypeptide.

In certain embodiments of the invention a fragment or variant of a naturally occurring polypeptide ligand or complement inhibitor is at least 70% identical, at least 80% identical, at least 90% identical, at least 95% identical, over an amino acid portion that constitutes at least 10%, at least 20%, at least 30%, at least 40%, at least 50%, at least 60%, at least 70%, at least 80%, or at least 90%, or 100% of the length of the naturally occurring counterpart. For example, variant that exhibits at least 50%, at least 60%, at least 70%, at least 80%, at least 90%, or greater sequence identity, over the relevant portion of the sequence could be used, wherein %identity is determined as described above. The amino acid portion is preferably at least 20 amino acids in length, more preferably at least 50 amino acids in length. Alternately, a fragment or variant can display significant or, preferably, substantial homology to a naturally occurring counterpart. Generally a fragment or variant of a naturally occurring polypeptide possesses sufficient structural similarity to its naturally occurring counterpart that it is recognized by an antibody (e.g., a polyclonal or monoclonal antibody) that recognizes the naturally occurring counterpart. Peptide ligands can be identified using phage display (Arap W, et al, Nature Medicine 8(2):121-7, 2002); Zurita A J, et al., J. Control Release, 91(1-2):183-6, 2003; Pasqualini, R. & Ruoslahti, E. Nature 380, 364-366, 1996; Pasqualini, R., et al., Trends Mol. Med. 8, 563-571, 2002).

In certain embodiments of the invention the ligand is an aptamer that binds to a cell type specific marker. In general, an aptamer is an oligonucleotide (e.g., DNA or RNA or) that binds to a particular protein or other target of interest. Aptamers are typically derived from an in vitro evolution process called SELEX and methods for obtaining aptamers specific for a target of interest are known in the art. See, e.g., Brody E N, Gold L. J Biotechnol. 2000 March; 74(1):5-13. Other methods can also be used.

Small molecules can also be used as ligands. Methods for identifying such ligands are known in the art. For example in vitro screening of small molecule libraries, including combinatorial libraries, and computer-based screening, e.g., to identify small organic compounds that bind to concave surfaces (pockets) of proteins, can identify small molecule ligands for numerous proteins of interest (Huang, Z., Pharm. & Ther. 86: 201-215, 2000).

In certain embodiments of the invention binding moieties are not proteins or molecules that are typically used as carriers and conjugated to antigens for the purpose of raising antibodies. Examples are carrier proteins or molecules such as bovine serum albumin, keyhole limpet hemocyanin, bovine gamma globulin, and diphtheria toxin. In certain embodiments of the invention the cell binding moiety is not an Fc portion of an immunoglobulin molecule.

Methods for covalently or noncovalently linking a complement inhibitor, e.g., a compstatin analog, to a binding moiety are known in the art. Suitable methods for linking two or more biomolecules are described in U.S. Ser. No. 10/923,940. General methods for conjugation and cross-linking are described in “Cross-Linking”, Pierce Chemical Technical Library, available at the Web site having URL www.piercenet.com and originally published in the 1994-95 Pierce Catalog and references cited therein, in Wong S S, Chemistry of Protein Conjugation and Crosslinking, CRC Press Publishers, Boca Raton, 1991; and G. T. Hermanson, supra. See also, Allen, T. M., Nature Reviews Cancer, 2, 750-763, 2002, which describes methods of making targeted therapeutic agents. For example, according to certain embodiments of the invention a bifunctional crosslinking reagent is used to couple a complement inhibitor, e.g., a compstatin analog, with an antibody or ligand. In general, bifunctional crosslinking reagents contain two reactive groups, thereby providing a means of covalently linking two target groups. The reactive groups in a chemical crosslinking reagent typically belong to various classes including succinimidyl esters, maleimides, pyridyldisulfides, and iodoacetamides. Bifunctional chelating agents may also be used.

Alternately, the complement inhibitor and the moiety can be produced as a fusion protein in certain embodiments of the invention.

Additional Modifications

Complement inhibitors, e.g., a compstatin analog, optionally linked to a binding moiety, can be modified by addition of a molecule such as polyethylene glycol (PEG) or similar molecules, e.g., the Fc domain of an antibody, to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458 (2002; Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476 (2002; Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures.

Therapy for Trauma and Kits

The invention encompasses administration of a complement inhibitor as described herein, optionally in conjunction with additional therapy. Such therapy may include administration of any active agent known in the art for treating trauma patients, such as blood products, recombinant factor VIIa or other clotting factors, blood substitutes, fluids, volume expanders, anti-hypotensive agents, antibiotics, etc. As noted above, the invention provides fixed dose formulations containing sufficient complement inhibitor to significantly inhibit complement activation in a child or adult human following a single intravenous (IV) administration (which may take the form of an IV bolus, IV infusion, IV drip in which a defined volume of fluid is administered, or combination thereof) or, in the case of orally bioavailable agents, oral administration. Such formulations may take the form of a physically discrete unit that contains a predetermined quantity of complement inhibitor. For example, such formulations could reduce systemic complement activity or activation by between 50% and 99%, e.g., by at least 50%, 75%, or 90%, relative to levels present prior to administration or relative to normal, average levels that would have been present in the absence of the complement inhibitor. In certain embodiments of the invention the formulation reduces the amount of systemic complement activation in a trauma patient by between 50% and 99%, e.g., by at least 50%, 75%, or 90%, relative to that which would have occurred in the absence of the complement inhibitor. The formulation may be a liquid formulation provided in a vial, a prefilled syringe, etc. One aspect of the invention is articles of manufacture containing a fixed dose of complement inhibitor in a convenient form for rapid administration, e.g., rapid IV administration, to a subject. In certain embodiments the complement inhibitor may be added directly to an IV fluid solution.

The invention provides a kit containing at least one fixed dose formulation comprising a complement inhibitor. Also provided are kits containing multiple fixed dose formulations of a complement inhibitor, wherein at least two of the fixed dose formulations contain different amounts of the complement inhibitor, wherein such different amounts are selected to achieve a desired amount of complement inhibition in, e.g., infants, children, and adults. In some embodiments fixed dose formulations containing three different amounts of a complement inhibitor are included. Emergency response vehicles, e.g., ambulances, and/or emergency rooms, may be equipped with such kits.

Pharmaceutical Compositions and Delivery Vehicles and Methods

Suitable preparations, e.g., substantially pure preparations of the complement inhibitor, e.g., compstatin analog or mimetic may be combined with pharmaceutically acceptable carriers, diluents, solvents, etc., to produce an appropriate pharmaceutical composition. Such preparations are an aspect of the invention.

In various embodiments of the invention an effective amount of the pharmaceutical composition is administered to a subject by any suitable route of administration including, but not limited to, intravenous, intramuscular, by inhalation, by nasal delivery (e.g., to the brain or systemically), by catheter, etc.

The term “pharmaceutically acceptable carrier or vehicle” refers to a non-toxic carrier or vehicle that does not destroy the pharmacological activity of the compound with which it is formulated. Pharmaceutically acceptable carriers or vehicles that may be used in the compositions of this invention include, but are not limited to, water, physiological saline, and the like.

The composition may include other components as appropriate for the formulation desired, e.g., buffer substances, anti-bacterial agents, etc. Supplementary active compounds, e.g., compounds independently useful for treating a trauma or surgery patient, can also be incorporated into the compositions.

It will be appreciated that the complement inhibitor and/or additional active agent can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Solutions or suspensions used for parenteral (e.g., intravenous) or intramuscular, administration can include the following components in various embodiments of the invention: a sterile diluent such as water for injection, saline solution, antibacterial agents such as benzyl alcohol or methyl parabens; antioxidants such as ascorbic acid or sodium bisulfite, chelating agents such as ethylenediaminetetraacetic acid; buffers such as acetates, citrates, lactates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or infusion bags or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. Sterile injectable solutions can be prepared by incorporating the complement inhibitor in the required amount in an appropriate solvent with one or a combination of ingredients such as buffers, antibacterial agents, etc., as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation include vacuum drying and freeze-drying which yields a powder of the active ingredient plus any additional desired ingredient from a previously sterile-filtered solution thereof.

In addition to the agents described above, in certain embodiments of the invention, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, polylactic acid, poly-lactic-glycolic acid (PLGA), etc. Methods for preparation of such formulations will be apparent to those skilled in the art. One of ordinary skill in the art will appreciate that the materials and methods selected for preparation of a controlled release formulation, implant, etc., should be such as to retain activity of the compound.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more elements or limitations found in any other claim that is dependent on the same base claim. Furthermore, where the claims recite a composition, it is to be understood that methods of administering the composition according to any of the methods disclosed herein, and methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. In addition, any particular embodiment, aspect, element, feature, etc., of the present invention may be explicitly excluded from the claims even if such exclusion is not set forth explicitly herein.

1-90. (canceled)

91. A method of selecting a trauma patient as a suitable candidate for therapy with a complement inhibitor, the method comprising:
(a) determining the genotype of the trauma patient with respect to one or more complement-related genes, wherein at least two alleles of the gene exist in the population, and wherein at least one allele is a risk allele for developing a complement-mediated disorder, which is optionally AMD; and
(b) selecting the trauma patient as a suitable candidate for therapy with a complement inhibitor based at least in part on the genotype.

92. The method of claim 91, wherein the trauma patient is selected as a suitable candidate if the trauma patient is homozygous or heterozygous for the risk allele.

93. The method of claim 91, wherein the trauma patient is selected as a suitable candidate if the trauma patient is homozygous for the risk allele.

94. The method of claim 91, wherein the trauma patient is selected as a suitable candidate if the trauma patient is not homozygous or heterozygous for the risk allele.

95. The method of claim 91, wherein the trauma patient is selected as a suitable candidate if the trauma patient is not homozygous for the risk allele.

96. The method of claim 91, wherein the gene encodes a complement protein.

97. The method of claim 91, wherein the gene encodes a complement control protein.

98. The method of claim 91, further comprising administering a complement inhibitor to the trauma patient.

99. The method of claim 91, further comprising administering a compstatin analog to the trauma patient.

100. The method of claim 91, wherein step (a) comprises determining the genotype with respect to two or more polymorphisms each of which is in or near a complement-related gene.

101-123. (canceled)

124. A method of identifying a trauma patient as being at increased risk of poor outcome following trauma, the method comprising:
(a) determining the genotype of the trauma patient with respect to one or more polymorphisms each of which is in or near a complement-related gene, wherein at least one allele of the polymorphism is a risk allele for a poor outcome following trauma (“trauma risk allele”); and
(b) identifying the patient as being at increased risk of poor outcome following trauma if the trauma patient's genotype comprises the trauma risk allele.

125. The method of claim 124, further comprising making a clinical decision based at least in part on whether the patient is identified as being at increased risk of poor outcome.

126. A complement inhibitor comprising a first cyclic peptide portion that inhibits C3 activation and a second cyclic peptide portion that comprises a C5a receptor antagonist.

127. The complement inhibitor of claim 126, wherein the first cyclic peptide portion comprises a compstatin analog and the second cyclic peptide portion comprises a sequence selected from SEQ ID NO: 57, 58, 77, and 78.

128. The complement inhibitor of claim 126, wherein the first cyclic peptide portion comprises a sequence selected from SEQ ID NO: 28, 29, 32, 34, 132, 133, and 135, and the second cyclic peptide portion comprises a sequence selected from SEQ ID NO: 57, 58, 77, and 78.
